(19) United States
(12) Patent Application Publication
(10) Pub. No.: US 2020/0199541 A1
(43) Pub. Date: Jun. 25, 2020

(54) BLOOD VESSEL ORGANOID, METHODS OF PRODUCING AND USING SAID ORGANOIDS

(71) Applicant: IMBA - INSTITUT FÜR MOLEKULARE BIOTECHNOLOGIE GMBH, Vienna (AT)

(72) Inventors: Josef PENNINGER, Vienna (AT); Reiner WIMMER, Vienna (AT); Dontscho KERJASCHKI, Vienna (AT)

(21) Appl. No.: 16/623,319

(22) PCT Filed: Jun. 15, 2018
(86) PCT No.: PCT/EP2018/065949

§ 371 Date: Dec. 16, 2019

(30) Foreign Application Priority Data
Jun. 16, 2017 (EP) 17176335.2

(51) Int. Cl. C12N 5/071; A61P 9/10

(52) U.S. Cl. CPC C12N 5/0691; A61K 45/06; A61P 9/10

(57) ABSTRACT

A method of generating an artificial blood vessel organoid, including providing stem cells capable of vascular differentiation, stimulating mesoderm differentiation in the stem cells, stimulating vascular differentiation in the stem cells, developing a cell aggregate from the stem cells, embedding the cell aggregates in a collagenous 3D matrix and stimulating vascular differentiation of the aggregate in the collagenous 3D matrix; organoids obtainable from the method, uses of the methods and organoids in manipulation and screening studied and kits for performing the methods.

### BACKGROUND OF THE INVENTION

Blood vessels are prone to various diseases called vascular diseases. Disorders in the network of blood vessels can cause a range of health problems which can be severe or prove fatal. Such diseases can be due to environmentally causes pathogenesis or due to developmental defects.

Goodwin (Microvasc Res. 2007; 74(2-3): 172-183) describes an in vitro angiogenesis assay in order to assess the activity of agents that affect angiogenesis in the pathogenesis of many dissases.

Duffy et al. (European Cells and Materials 21, 2011: 15-30) describes in vitro vascularisation of collagen-glycosaminoglycan scaffolds in a surface adherent 2D culture.

Nakagami et al. (Hypertension 2006; 48:112-119) provide a method of vascularization by cell-matrix interaction using matrigel that forced embryonic stem cells into development of sprouting blood vessels containing endothelial and vascular smooth muscle cells.

Kusuma et al. (PNAS 110(31), 2013: 12601-12606), Gerecht-Nir et al. (Laboratory Investigation 83(12), 2003: 1811-1820), WO2007/140340 A2, WO2014/145871 A1, US 2014/273220 A1 and WO2017/015415 A1 describe the formation of vascular structures out of isolated early vascular cells in an engineered matrix. Before introduction into the matrix, the cells may be derived from human pluripotent stem cells that differentiated to early vascular cells and then individualized (by trypsinization and/or 40 μm mesh filtration). The individual cells grow in the matrix and assemble there to form vascular networks. The goal in these papers was to provide self-assembling cells that are useful in regenerative medicine.

WO2011/115974 A1 relates to a device to form 2D cultured vascular networks on a surface.

Shen et al. (Cell Research 13 (5) (2003): 335-341) describes the formation of enginerred blood vessels from adult rabit smooth muscle cells and differentiated endothelial cells from mouse.

Still, previous vascular models lack sufficient similarity with natural in vivo formed vascular networks and there is a need for improved, more life-like vascular models.

Therefore, it is a goal of the present invention to provide improved vascular models, in addition also models that allow broader varieties of uses, such as disease models and testing in screening procedures.

### SUMMARY OF THE INVENTION

The present invention provides a method of generating an artificial blood vessel organoid, comprising providing stem cells capable of vascular differentiation, stimulating mesoderm differentiation in said stem cells, stimulating vascular differentiation in said stem cells, developing a cell aggregate from said stem cells, embedding said cell aggregate in a collagenous 3D matrix and stimulating vascular differentiation of the aggregate in said collagenous 3D matrix.

In a closely related aspect, the invention provides a method of generating an artificial blood vessel organoid, comprising embedding vascular stem cells in a collagenous 3D matrix comprising 10%-50% laminin, 20%-70% collagen I, and/or 2%-30% collagen IV and stimulating vascular differentiation of said stem cells in said collagenous 3D matrix.

Such methods can be used to provide a blood vessel organoid, that forms a further aspect of the invention. In particular, the invention provides an artificial blood vessel organoid culture comprising an interconnected network of vascular capillaries, said capillaries comprising endothelium and a basal membrane with peri-vascular pericytes, (i) wherein said organoid is produced by a method of the invention and/or (ii) wherein the capillaries are embedded in an artificial 3D matrix comprising a hydrogel with collagen and/or (iii) wherein the organoid culture comprises 40 to 1000 blood vessels as counted by counting individual vessels and vessels between capillary intersections. All three characteristics (i), (ii) and (iii) are hallmarks of the invention, that can be individually required by an inventive artificial blood vessel organoid culture or in combination.

The invention further provides a method of providing a non-human animal model with human vascular capillaries, wherein said human capillaries comprise endothelium and a basal membrane with perivascular pericytes, comprising the steps of introducing a human blood vessel organoid of the invention into a non-human animal and letting said organoid grow its vascular capillaries.

The invention also relates to a non-human animal model comprising such an artificial blood vessel organoid culture, e.g. as an insert. Furthermore, a non-human animal model with human vascular capillaries is provided, wherein said human capillaries comprise endothelium and a basal membrane with perivascular pericytes.

The invention further relates to the use of the inventive culture or non-human animal model or the method in generating them as model of a pathology, e.g. diabetes, wherein the cells in the method, the organoid or the organoid in the non-human animal model are subject to pathogenesis to develop said pathology, e.g. hyperglycemia or destruction of pancreatic beta-cells in case of diabetes.

The invention further provides a method of screening a candidate chemical compound for influencing a pathogenesis or a pathology comprising administering said candidate chemical compound to a culture or non-human animal model or during generation of said culture or non-human animal model according to any aspect of the invention and monitoring for physiological differences in said culture or animal model as compared to said culture or animal model without administration of the candidate chemical compound.

The invention has provided new treatment models for diabetes. Accordingly, the invention provides a use of a Notch3 activation pathway inhibitor (such as a gamma-secretase inhibitor, a Notch3 inhibitor, DLL4 inhibitor or a combination thereof) in the treatment or prevention of a thickened capillary basement membrane, such as in diabetic vasculopathy, occlusive angiopathy, altered vascular permeability, tissue hypoxia, heart disease, stroke, kidney disease, blindness, impaired wound healing or chronic skin ulcers. Also provided is a Notch3 activation pathway inhibitor (e.g. gamma-secretase inhibitor, a Notch3 inhibitor, DLL4 inhibitor) for use in such a treatment or prevention; or the use of a Notch3 activation pathway inhibitor (e.g. gamma-secretase inhibitor, a Notch3 inhibitor, DLL4 inhibitor) for use in manufacturing a medicament or pharmaceutical composition for such a treatment or prevention.

Finally, the invention provides a kit suitable for the generation of an artificial blood vessel organoid according to any inventive method, comprising (i) a Wnt agonist or a GSK inhibitor; (ii) a vascular differentiation factor selected from VEGF, a FGF, a BMP; (iii) a collagenous 3D matrix.

All embodiments of the invention are described together in the following detailed description and all preferred embodiments relate to all embodiments, aspects, methods, organoids, animal models, uses and kits alike. E.g. kits or their components can be used in or be suitable for inventive methods. Any component used in the described methods can be in the kit. Inventive organoids are the results of inventive methods or can be used in inventive methods and uses. Preferred and detailed descriptions of the inventive methods read alike on suitability of resulting or used organoids or animal models of the inventions. All embodiments can be combined with each other, except where otherwise stated.

### DETAILED DESCRIPTION OF THE INVENTION

The present invention provides a method of generating an artificial blood vessel organoid. Such artificial organoids are in vitro grown but highly resemble in vivo capillary structures. An organoid is a miniaturized and simplified version of an organ produced in vitro in three dimensions that shows realistic micro-anatomy. They are derived from one or a few cells from a tissue, embryonic stem cells or induced pluripotent stem cells, which can self-organize in three-dimensional culture owing to their self-renewal and differentiation capacities.

The inventive organoids derived from human stem cells recapitulate the structure and function of human blood vessels. 3D blood vessel organoids from embryonic and induced pluripotent stem cells are provided. These blood vessel organoids contain endothelium, perivascular pericytes, and basal membranes, and self-assemble into lumenized interconnected capillary networks. Human blood vessel organoids transplanted into mice form a perfused human vascular tree, including human arterioles and venules. Intriguingly, exposure of blood vessel organoids to hyperglycemia and inflammatory cytokines in vitro induced thickening of the basal membrane and transcriptional changes in endothelial cells, mimicking the microvascular changes in diabetic patients. A drug screen uncovered a y-secretase inhibitor that attenuated this “diabetic” vasculopathy in blood vessel organoids. Blood vessel organoids can be used to generate disease models for drug discovery, as we have shown by identifying y-secretase as a potential therapeutic target for diabetic vasculopathy, which affects hundreds of millions of patients.

The method of generating such organoids comprises the steps of providing stem cells capable of vascular differentiation, stimulating mesoderm differentiation in said stem cells, stimulating vascular differentiation in said stem cells, developing a cell aggregate from said stem cells, embedding said cell aggregate in a collagenous 3D matrix and stimulating vascular differentiation of the aggregate in said collagenous 3D matrix.

Stem cells capable of vascular differentiation are for example pluripotent stem cells. Pluripotent stem cells may be derived from embryonic stem cells or they may be induced pluripotent stem cells (iPS). iPS are preferred.

The stem cells are differentiated into a mesodermal-vascular route. Differentiation can be achieved by contacting cells with a tissue (mesodermal/vascular) specific growth or differentiation factor. The cells may then develop into the desired tissue. Such a tissue specific growth or differentiation factor may be a mesoderm and/or a vascular differentiation factor, preferably used at different stages of the inventive method. This will determine the development into the respective type of cellular tissue in later development. The cells will thereby transit from pluripotent to multipotent cells. Other tissue types shall then be not or only by a return to a pluripotent status be possible again. Usually not all cells are differentiated to the selected tissue type. It usually sufficient when about 50% or more or at least 55% or at least 60%, or at least 65% or at least 70% or at least 75% of the cells initiate differentiation towards the selected tissue type (in particular mesoderm) and transform to reduce their differentiation potential by multipotent cell with the respective tissue destiny (%-values as fractions of the cell amount) at first. Of course, this differentiation destiny only applies for the cells that are not returned to a un- or less differentiated state by use of artificial growth and dedifferentiation stimuli. Clearly, even somatic cells can be returned to pluripotent cells and this is not meant when defining a differentiated state herein. Preferably, no factors are introduced to the cells that would return the cells to pluripotent cells once the mesoderm or vascular differentiation is initiated.

The inventive organoids can be obtained from culturing pluripotent stem cells. In principle, the cells may also be totipotent, if ethical reasons allow.

A “totipotent” cell can differentiate into any cell type in the body, including the germ line following exposure to stimuli like that normally occurring in development. Accordingly, a totipotent cell may be defined as a cell being capable of growing, i.e. developing, into an entire organism.

The cells used in the methods according to the present invention are preferably not totipotent, but (strictly) pluripotent.

In a particular preferred embodiment, the cells of the present invention (including all further embodiments related thereto), are pluripotent.

A “pluripotent” stem cell is not able of growing into an entire organism, but is capable of giving rise to cell types originating from all three germ layers, i.e., mesoderm, endoderm, and ectoderm, and may be capable of giving rise to all cell types of an organism. Pluripotency can be a feature of the cell per see, e.g. in certain stem cells, or it can be induced artificially. E.g. in a preferred embodiment of the invention, the pluripotent stem cell is derived from a somatic, multipotent, unipotent or progenitor cell, wherein pluripotency is induced. Such a cell is referred to as induced pluripotent stem cell herein. The somatic, multipotent, unipotent or progenitor cell can e.g. be used from a patient, which is turned into a pluripotent cell, that is subject to the inventive methods. Such a cell or the resulting organoid culture can be studied for abnormalities, e.g. during organoid culture development according to the inventive methods. A patient may e.g. suffer from a vascular disorder. Characteristics of said disorder can be reproduced in the inventive organoids and investigated.

A “multipotent” cell is capable of giving rise to at least one cell type from each of two or more different organs or tissues of an organism, wherein the said cell types may originate from the same or from different germ layers, but is not capable of giving rise to all cell types of an organism.

In contrast, a “unipotent” cell is capable of differentiating to cells of only one cell lineage.

A “progenitor cell” is a cell that, like a stem cell, has the ability to differentiate into a specific type of cell, with limited options to differentiate, with usually only one target cell. A progenitor cell is usually a unipotent cell, it may also be a multipotent cell.

With decreasing differentiation capabilities, stem cells differentiate in the following order: totipotent, pluripotent, multipotent, unipotent. During development of the inventive organoid, stem cells differentiate from pluripotent (also totipotent cells are possible) into multipotent mesoderm, vascular or endothelial stem cells, further into unipotent stem cells of endothelial cells and pericytes.

Preferably, the stem cell is from a vertebrate, such as a mammal, reptile, bird, amphibian or fish. In particular preferred are land-living vertebrates. Possible are non-human animals and humans. Especially preferred are mammals for all aspects and embodiments of the invention, such as mouse, cattle, horses, cats, dogs, non-human primates; human cells are most preferred. The non-human animal model comprising the organoid may be selected from the same animals. The stem cells and the animal model may not be the same organism.

Differentiation of stem cells has become a standard technique in the art. For example, differentiation and growth factors to form vascular grafts are disclosed in WO2016/094166 A1. Such growth factors can also be used according to the invention as differentiation factors.

The inventive method comprises a step of inducing mesoderm differentiation. There are differentiation stimuli, that drive differentiation specifically into one direction (e.g. mesoderm) and those that drive unspecific differentiation with mesoderm being among several other differentiation routes. Such unspecific differentiation can be achieved by serum, such as FBS (fetal bovine serum) as used in Gerecht-Nir et al. (see background section). Unspecific differentiation may lead do various germ layer being present, including ectoderm, including neuroectoderm and endoderm.

According to a preferment of the invention specific mesoderm differentiation is performed, such as by a mesoderm specific differentiation factor. Alternatively, but less preferred, mesoderm can be selected from the differentiated cells. Selection can be combined with specific differentiation stimulation. Selection of cells is not desired because it would require isolation and individualization of cells. According to the invention, such individualization is disadvantageous because the cells should form or start forming an aggregate at this stage. Preferably, the cells after mesoderm stimulation have at least 50%, preferably at least 60%, even more preferred at least 70% or even at least 80%, of its cells in mesoderm differentiation. Preferably, mesoderm differentiation comprises treating the stem cells with a Wnt agonist or a GSK inhibitor, preferably CHIR99021. Wnt agonist or a GSK inhibitor achieves a high rate of mesoderm differentiation. A Wnt agonist may be a Wnt stimulator like CHIR99021

The stem cells are also treated by vascular differentiation. Vascular differentiation is continuously or repeatedly stimulated in the inventive method, in particular within the 3D matrix but also before that aggregate of cells is introduced into the 3D matrix, when when the cells are forming said aggregate.

Vascular differentiation may comprise an endothelial differentiation and results in small capillary or capillary precursor formation. In early stages of the method, e.g. before the 3D matrix treatment, such endothelial/vascular differentiation may not lead to the same well-defined and life-like capillaries that will alter form in the 3D matrix.

As with mesoderm differentiation, preferably the vascular differentiation is a specific vascular differentiation, with preferably at least 50%, preferably at least 60%, even more preferred at least 70% or even at least 80%, of its cells in vascular differentiation. Preferably vascular differentiation in said stem cells comprises treating the stem cells with a VEGF and/or a FGF and/or a BMP and/or low oxygen conditions of 12% (v/v) or less atmospheric oxygen. VEGF, FGF, BMP and low oxygen may be combined. A preferred VEGF is VEGF-A. A preferred FGF is FGF-2. A preferred BMP is BMP4. Low oxygen conditions are 12% (v/v) or less atmospheric oxygen, i.e. oxygen in a gas phase supplied to the cells. The gas phase is preferably at atmospheric pressure. Preferably the oxygen content is even less, preferably 10% or less, more preferred 8% or less, e.g. 6% or less (all % in v/v). The oxygen content is preferably 2% or more, e.g. 2% to 12% (all % in v/v). Preferably, the cells are cultured in a medium with VEGF in a concentration of 10 ng/ml to 50 ng/ml, preferably about 30 ng/ml. Preferably, the cells are cultured in a medium with FGF in a concentration of 10 ng/ml to 50 ng/ml, preferably about 30 ng/ml. Preferably, the cells are cultured in a medium with BMP in a concentration of 10 ng/ml to 50 ng/ml, preferably about 30 ng/ml.

The stem cells before introduction into the 3D matrix are forming the aggregate of cells. Preferably these cells are in suspension culture that allows such aggregation. This means that the stem cells that are treated for mesoderm and/or vascular differentiation are already in small aggregates. Such aggregates are usually small to be able to be suspended in suspension culture in a liquid culture medium, without a stable 3D matrix.

After the differentiation, before embedding the differentiated stem cells, now in an aggregate, in to the 3D matrix usually at least 30%, preferably at least 40%, e.g. about 50% of the cells of the aggregate are endothelial cells. Preferably at least 20%, e.g. 30% of the cells of the aggregate are pericytes. Together, preferably at least 60%, preferably at least 70%, e.g. about 80% of the cells are vascular cells.

Once the aggregates are embedded in the 3D matrix, the inventive method also comprises vascular differentiation of the aggregate in said 3D matrix. Preferably also this vascular differentiation comprises specific vascular differentiation. Especially preferred vascular differentiation of the aggregate comprises treating cells of the aggregate with a VEGF and/or a FGF. A preferred VEGF is VEGF-A. A preferred FGF is FGF-2. Preferably, the aggregates in the matrix are cultured in a medium with VEGF in a concentration of 60 ng/ml to 150 ng/ml, preferably about 100 ng/ml. Preferably, the aggregates in the matrix are cultured in a medium with FGF in a concentration of 60 ng/ml to 150 ng/ml, preferably about 100 ng/ml.

The cell aggregate that formed from the stem cells after culturing, especially during mesoderm and/or vascular differentiation, is embedded into the 3D matrix. This aggregate that is embedded into the 3D matrix preferably has a size of at least 30 cells or at least 50 cells, preferably of at least 100 cells, especially preferred at least 300 cells, e.g. about 1000 cells. Preferably the size is less than 100000 cells, e.g. less than 30000 cells. The cell aggregate should have an established size but not too large to suffer from low stability in liquid suspension cultures. An aggregate is an accumulation of cells attached to each other, in particular by intercellular bonds and intercellular connections.

Preferably, said aggregate is embedded in the collagenous 3D matrix at day 7 to 15 from the start of aggregate formation. In this time, the aggregate usually has a suitable size and differentiation status. A preferred time-line is shown in FIG. 1a. Preferably, mesoderm differentiation stimulation (mesoderm induction) is at days 2-6, preferably vascular differentiation stimulation (vascular lineage promotion) is at days 4-14.

Embedding the cells into the 3D matrix can be performed by any method known in the art. A preferred method is fluidizing 3D matrix material and solidifying or gelling the 3D matrix around the aggregate of cells.

The 3D matrix is a collagenous matrix, it has preferably at least 50 wt.-% collagen. Collagen includes collagen I, collagen II, collagen III and collagen IV. Collagen I and collagen IV are most preferred. Preferably the at least 50% is composed of collagen I or collagen IV, most preferably a mixture of collagen I and collagen IV.

The aggregate is cultured in the three dimensional (3D) matrix. A 3D matrix is distinct from 2D cultures, such as 2D cultures in a dish on a flat surface. A “3D culture” means that the culture can expand in all three dimensions without being blocked by a one-sided wall (such as a bottom plate of a dish). Such a culture, preferably including the 3D matrix, is preferably in suspension. The 3D matrix may be a gel, especially a rigid stable gel, which results in further expansion of growing cell culture/tissue and differentiation. The gel may be a hydrogel. A suitable 3D matrix according to the invention comprises collagen. More preferably the 3D matrix comprises extracellular matrix (ECM) or any component thereof selected from collagen, lam inin, entactin, and heparin-sulfated proteoglycan or any combination thereof. Extra-cellular matrix may be from the Engelbreth-Holm-Swarm tumor or any component thereof such as laminin, collagen, preferably type 4 collagen, entactin, and optionally further heparan-sulfated proteoglycan or any combination thereof. Such a matrix is Matrigel. Matrigel is known in the art (U.S. Pat. No. 4,829,000) and has been used to model 3D heart tissue previously (WO 01/55297 A2) or neuronal tissue (WO 2014/090993). Preferably the matrix comprises laminin, collagen and entactin, preferably in concentrations 20%-85% laminin, 3%-50% collagen and sufficient entactin so that the matrix forms a gel, usually 0.5%-10% entactin. Laminin may require the presence of entactin to form a gel if collagen amounts are insufficient for gel forming. A Matrigel-rich matrix may comprise a concentration of at least 3.7 mg/ml containing in parts by weight about 50%-85% laminin, 5%-40% collagen IV, optionally 1%-10% nidogen, optionally 1%-10% heparan sulfate proteoglycan and 1%-10% entactin. According to the invention, the collagen content is preferably increased, in particular preferred by collagen I. A particularly preferred matrix of the present invention in all embodiments comprises 10%-50% laminin, 20%-70% collagen I, and/or 2%-30% collagen IV; preferably further 0.5%-10% nidogen, 0.5%-10% heparan sulfate proteoglycan, and/or 0.5%-10% entactin (all wt.-%). These percentages relate to the solid, proteinaceous components only, i.e. not liquid components like water, which may me the major component in a hydrogel. Matrigel's solid components usually comprise approximately 60% laminin, 30% collagen IV, and 8% entactin. The 3D matrix may be a mixture of Matrigel and collagen, e.g. a mixture 2:1 to 1:3, preferably of about 1:1 of matrigel:collagen I. All %-values given for the matrix components are in wt.-%. These values may vary, e.g. by +/−30% depending on the source. Entactin is a bridging molecule that interacts with laminin and collagen. Such matrix components can be added in step r). These components are also preferred parts of the inventive kit. The 3D matrix may further comprise growth factors, such as any one of EGF (epidermal growth factor), FGF (fibroblast growth factor), NGF, PDGF, IGF (insulin-like growth factor), especially IGF-1, TGF-β, tissue plasminogen activator. The 3D matrix may also be free of any of these growth factors.

In general, the 3D matrix is a three-dimensional structure of a biocompatible matrix. It preferably comprises collagen, gelatin, chitosan, hyaluronan, methylcellulose, laminin and/or alginate. The matrix may be a gel, in particular a hydrogel. Organo-chemical hydrogels may comprise polyvinyl alcohol, sodium polyacrylate, acrylate polymers and copolymers with an abundance of hydrophilic groups. Hydrogels comprise a network of polymer chains that are hydrophilic, sometimes found as a colloidal gel in which water is the dispersion medium. Hydrogels are highly absorbent (they can contain over 99 wt.-% water) natural or synthetic polymers. Hydrogels also possess a degree of flexibility very similar to natural tissue, due to their significant water content. It is possible that the three dimension-al matrix, or its components, especially ECM or collagen, still remains in the produced tissue culture. Preferably the 3D matrix is a collagenous matrix, preferably it contains type I and/or type IV collagen.

Preferably the 3D matrix is a hydrogel. The matrix, in particular the hydrogel may have a viscoelastic storage modulus G′ of 10 to 30. The storage modulus in viscoelastic materials measure the stored energy, representing the elastic portion, and the energy dissipated as heat, representing the viscous portion. A method to determine the storage modulus, which can be used according to the invention, e.g. by rheometer, is given in Angui ano et al. PLoS ONE 2017, 12(2): e0171417.

Preferably the collagenous 3D matrix comprises 10%-50% laminin, 20%-70% collagen I, and/or 2%-30% collagen IV; preferably further 0.5%-10% nidogen, 0.5%-10% heparan sulfate proteoglycan, and/or 0.5%-10% entactin (all wt.-o). Matrigel usually comprises 50%-85% laminin, 5%-40% collagen IV, 1%-10% nidogen, 1%-10% heparan sulfate proteoglycan and 1%-10% entactin (solid, proteinaceous components only).

The invention also provides a method of generating an artificial blood vessel organoid, comprising embedding vascular stem cells in a collagenous 3D matrix comprising 10%-50% laminin, 20%-70% collagen I, and/or 2%-30% collagen IV and stimulating vascular differentiation of said stem cells in said collagenous 3D matrix. All aspects and preferred embodiments discussed so far also apply to this method, that also forms an independent aspect of the invention. It has been shown herein, that such a 3D matrix results in very favourable vascular networks that are reminiscent of in vivo vascular networks. In particular, such networks have large lumen and are capable of incorporation into model animals with connection to the circulatory system of the model animals. Preferably the vascular stem cells are generated by differentiating mesodermal stem cells into vascular stem cells, preferably wherein the mesodermal stem cells have been obtained by stimulating mesodermal differentiation in pluripotent stem cells, in particular, as mentioned above. All of these aspects are combinable with the above description.

In all embodiments and aspects of the invention, preferably the cells of the aggregate are cultured in said 3D matrix for at least 5 days, preferably for at least 7 days. Culturing in the 3D matrix may be for 5 to 60 days or more, preferably for at least 10 days.

The 3D matrix itself may be suspended in suspension culture.

In the 3D matrix, the aggregates form vascular networks, comprising an endothelium, formed by endothelial cells, surrounded by perivascular pericytes forming a basal membrane, which is further described in the following. The self-assembly of vascular networks usually occurs through sprouting angiogenesis by sprouting of vessels into the matrix.

The invention further provides an artificial blood vessel organoid culture comprising an interconnected network of vascular capillaries, said capillaries comprising endothelium and a basal membrane with perivascular pericytes, (i) wherein said organoid is produced by a method of the invention and/or (ii) wherein the capillaries are embedded in an artificial 3D matrix comprising a hydrogel with collagen and/or (iii) wherein the organoid culture comprises 40 to 1000 blood vessels as counted by counting individual vessels and vessels between capillary intersections. All three characteristics (i), (ii) and (iii) are hallmarks of the invention, that can be individually required by an inventive artificial blood vessel organoid culture or in combination. The organoid may still comprise the 3D matrix or parts of it (ii). The same as described above for the 3D matrix in the method applies to the organoid.

The organoid is considered an artificial tissue. “Artificial” means that it is grown in vitro and has certain characteristics of artificial cultures, like size, consistency, shape and cell organization. The shape may be irregular and different from natural occurring tissues and cell organization may differ due to size restrains. In particular, “artificial” excludes naturally occurring tissues and organs and their parts, e.g. natural tissue slices. The 3D matrix may be still in the culture and/or the organoid may have the shape determined by growth in such a matrix. E.g. the organoid may be obtainable by growth in a 3D matrix, especially those as described above. The artificial organoid culture is in particular not a culture of an in vivo developed vascular system or a tissue sample thereof.

The number of blood vessels in the organoid is surprisingly high and has not been achieved before in an artificial culture (iii). Preferably, the organoid culture comprises at least 40, even more preferred at least 60, at least 100, at least 200 or at least 300 or more blood vessels as counted by counting individual vessels and vessels between capillary intersections. The upper value of 1000 capillaries is a result of a usual organoid that still has a small manageable size, e.g. for screening methods in cell culture well plates, but of course even larger sizes and capillary numbers are possible by continuing the organoid cultivation. Capillary numbers are counted as is usually in the field, i.e. by counting individual vessels and vessels between capillary intersections. The number may be inferred from counting in a small portion of the organoid and extrapolating the number to the entire organoid.

Preferably, the vascular capillaries of the artificial blood vessel organoid culture have an average diameter of from 1 μm to 30 μm, preferably 5 μm to 20 μm. Such large diameters and volumes of the capillaries allow perfusion in a circulatory animal model. Preferably, the average diameter of vascular capillaries is at least 1 μm, more preferred at least 2 μm, even more preferred at least 3 μm, at least 4 μm, at least 5 μm, at least 6 pm or more.

The artificial blood vessel organoid preferably has a size of 100 μm to 10 mm in its longest dimension. Preferred is as size of 250 μm to 10 mm or 500 μm to 5 mm. This size is of the organoid itself, i.e. the culture comprising the entire vascular network, preferably the organoid is still in the 3D matrix.

Such a size, especially of about 1-2 mm, makes the organoids manageable for cell culture well plates, such as 96 well plates, that may be used for large-scale testing purposes. The organoids are stable and can endure physical stress that allows transportation, e.g. by pipette and they are thus suitable for routine lab handling or automated processing in a screening robot.

The artificial blood vessel organoid may be provided in form of a globular body, e.g. in particular with the shortest dimension being not less than 20% of the longest dimension, in particular not less than 30% or not less than 40% of the longest dimension. Preferably the volume of the artificial blood vessel organoid is at least 1×10⁶ μm³, in particular preferred at least 2×10⁶ μm³, at least 4×10⁶ μm³, at least 6×10⁶ μm³, at least 8×10⁶ μm³, at least 10×10⁶ μm³, at least 15×10⁶ μm³ and/or sizes of at least 250 μm, especially preferred at least 350 μm.

The organoids may also be provided in discs, which may be suspended in a free floating environment for easy handling.

The presence of sufficient perivascular pericytes in the inventive artificial organoid was particularly surprising and shows that the inventive vascular network in the organoid has achieved in vivo characteristics. Perivascular pericytes support the endothelial cells. The ration of endothelial cells and perivascular pericytes may vary, dependent on organoid culturing time. Preferably, the ratio of endothelial cells to perivascular pericytes in the artificial blood vessel organoid culture is between 100:1 to 1:10. Preferably, the ratio is 50:1 to 1:5, or 25:1 to 1:4 or 10:1 to 1:3 or 5:1 to 1:2. Usually, in young organoids, the endothelial cells are in excess, in older organoids the ratio may be about 1:1: or even result in a pericyte excess in relation to endothelial cells.

Preferably the vascular capillaries of the artificial blood vessel organoid culture comprise mature endothelial cells. The mature endothelial cells may be reactive to TNF-alpha by responding by ICAM-1 expression. Preferably the vascular capillaries of the artificial blood vessel organoid culture comprise mature pericytes. Maturity of the pericytes may be detected by determining expression markers of mature pericytes.

The endothelial cells may be surrounded by a basal membrane (also referred to as basement membrane). The basal membrane may comprise collagen IV, fibronectin and/or laminin; it may be rich in collagen IV. Basal membrane thickness may be a marker for health of the capillaries and may be determined as an indicator in screening or other testing methods. The basal membrane of the vascular capillaries of the artificial blood vessel organoid culture may have a thickness in the range of 0.1 μm to 3 μm, preferably 0.3 μm to 2.5 μm, dependent on the size of the capillaries. Preferably the average thickness of the basal membrane of the vascular capillaries of the artificial blood vessel organoid is 0.3 μm to 2.5 μm, preferably 0.6 μm to 2.1 μm, especially preferred 0.8 μm to 1.8 μm, most preferred about 1.2 μm.

“About” means in this case +/−30%.

A further hallmark of the invention is that the organoids develop venules and arterioles as found in an in vivo vascular tree.

The invention further provides a method of providing a non-human animal model with human vascular capillaries, wherein said human capillaries comprise endothelium and a basal membrane with perivascular pericytes, comprising the steps of introducing a human blood vessel organoid of the invention, in particular as described above, into a non-human animal and letting said organoid grow its vascular capillaries. Preferably, said human organoid is introduced onto or into the kidney of the non-human animal. The invention also provides a non-human animal model comprising an inserted artificial blood vessel organoid culture according to the invention. As said, an advantage of the inventive organoid is its versatility and in vivo-like structure of vascular capillaries. It is possible to study the behaviours of these capillaries in vivo by introduction into a non-human animal.

Animal models to study and investigate vascular diseases, such as diabetes have been known in the art (e.g. WO 2015/044339 A1). These models usually are based on an animal that has a genetic alteration that gives rise to a diseased state. However, such mutations also alter the study premise and potentially the alters not just disease occurrence but also the response to any tested treatment options. Therefore, there is a need to study life-like situations. Especially preferred is a model of human vascular systems. The present invention achieves that goal by providing an organoid that is suitable for implantation in a test animal. As described above, the inventive organoid, that is grown in vitro has a high resemblance of vascular networks formed in vivo, however, in an artificial and controllable environment (e.g. still in the 3D matrix instead of connective tissue). Among the properties of the inventive vascular system, that can now be introduced in a non-human anima, are capillaries with endothelium and a basal membrane with perivascular pericytes. Therefore, the invention also provides a non-human animal model with human vascular capillaries, wherein said human capillaries comprise endothelium and a basal membrane with perivascular pericytes. All these kinds of animal models are described together and each preferred or further embodiment reads on all inventive animal models.

The invention now allows studying human vascular capillaries in non-human animals. Therefore, preferably the organoid is from human cells, i.e. having human vascular capillaries, in a non-human animal. The non-human animal is preferably a vertebrate, e.g. a mammal, reptile, bird, amphibian or fish. In particular preferred are land-living vertebrates. Especially preferred are mammals for all aspects and embodiments of the invention, such as mouse, cattle, horses, cats, dogs, non-human primates. Of course, also any vertebrate may be used as source for the organoid and hence the vascular capillaries. But of course, the remarkable advantages are associated with human organoids giving that it is also possible to create non-human animals as model organisms without having to use an organoid. Prefearly, the non-human animal is immunocompromised in order to avoid organoid rejection.

Contrary to previous non-human animals comprising human endothelial cells as summarized in the background section, the present invention not only introduces human cells into non-human animals but introduces fully developed capillary systems of a human into the non-human animal. I.e. human endothelial is investigated in the surrounding of human basal membrane and human pericytes in a structure reminiscent of human capillary trees, in particular a capillary system comprising venules and arterioles.

Preferably the vascular capillaries of the artificial blood vessel organoid culture or the human vascular capillaries in the animal model are perfused by the blood circulatory system of the non-human animal. As said, it is one of the advantages of the invention that the capillaries of the organoid are capable to connect to the vascular system of the non-human animal model. Such connections will form once implanted into a suitable location in the animal. A very reactive location is the kidney membrane but other locations are suitable as well as is known in the art for tissue transplant and trans-grafting studying techniques. Other organs may be used, such as any organ in the abdominal cavity or by subcutaneous transplantation. In certain cases, a given location may require further stimulation of capillary growth, such as by supplying growth factors, e.g. in a suitable matrix, like a hydrogel or sponge.

The inventive artificial blood vessel organoid culture can also be used as a research tool to study the effects of any external (e.g. drugs or other stimuli) or internal (mutations) influences on growth and activity of cells in the organoid. Therefore, in an additional aspect, the invention provides a method of investigating a developmental vascular tissue effect, e.g. a defect, in particular a developmental defect, comprising (i) decreasing or increasing the expression in a gene of interest in a cell at any stage during the inventive method or to the developed (finished) organoid or animal model, or (ii) administering a candidate chemical compound of interest to a cell during development of the organoid at any stage during the inventive method or to the developed (finished) organoid or animal model. A gene of interest can be a gene, that is suspected to be essential or detrimental when active during the development healthy vascular tissue. Preferred genes are genes that are associated with a disease, for example genes that are causative agents in genetic diseases. Methods to decrease or increase expression in a gene are well known in the art, and include knock-out, knock-down methods or mutagenesis (especially RNA interference, anti-sense inhibition, shRNA silencing, mutagenesis by CRISPR-Cas, etc.), or introductions of transgenes (e.g. knock-in), respectively. Such decrease or increases can be conditional, e.g. by introducing a genetic construct with inducible promoters and/or conditional knock-out or knock-downs or knock-ins. The introduction of conditional mutations of essential genes or introductions of lethal genes are possible by using suitable conditional mutation vectors, e.g. comprising a reversible gene trap. Conditional mutations preferably facilitate reversible mutations, which can be reversed to a gene-active or inactive, respectively, state upon stimulation, e.g. as in the double-Flex system (WO 2006/056615 A1; WO 2006/056617 A1; WO 2002/88353 A2; WO 2001/29208 A1). Mutations can either be random or site-directed at specific genes. Thus in preferred embodiments of the invention, reversible mutations are introduced into the pluripotent stem cells, either by random (forward) or site directed (reverse) mutagenesis. Suitable vectors comprising insertion cassette with a reversible mutations. Mutations can be switched on or off at any stage of the inventive method. Vectors or other nucleic acids can be introduced into cells with any method known in the art, e.g. electroporation. It is of course also possible to provide cells having a given mutation. Such cells can be isolated from a patient, followed by a step of inducing pluripotent stem cell status, and letting the cells develop into the inventive tissue, e.g. by the method described above. The patient may have a particular disease of interest, especially a vascular defect or capillary deformity. Candidate chemical compounds are further explained below with regard to candidate therapeutic potential. However, any candidate compound can also be assayed for any desired effect to the cells, capillaries or the entire organoid. Preferred candidate compounds are small organic molecules.

In any method or culture or non-human animal model of the invention, preferably the blood vessels or capillaries are subjected to pathogenesis and said organoid or human animal model is a model of a pathology.

The inventive capillary formation process may be subject to a disorder; alternatively, a pathological state may be induced in the organoid as such, e.g. in a culture, or as implant in the animal model.

Induction of a pathology for research purposes is known in the art and may be exposure to harmful compounds or pathogens, a disadvantageous diet or mechanical stress or injury or a combination of these (e.g. as disclosed in US 2010/124533 A1). Pathogens include microorganisms, in particular bacteria or fungi and viruses. A pathology may also be the result of a genetic disorder or malfunction.

Pathogenesis may comprise hyperglycaemia and/or inflammation. Both can be found in diabetes, for example. In particular preferred, the pathology is diabetes. Inflammation can comprise exposure to or induction of one or more inflammatory cytokines, preferably TNF-alpha and/or IL-6. Hyperglycaemia means increased glucose levels reminiscent of diabetes type 2. Such glucose levels may e.g. be at least 50 mM, preferably at least 70 mM. Example conditions to induce diabetes are 75 mM D-Glucose+1 ng/mL TNF-α+ing/mL IL-6 for 1-2.5 weeks. Diabetic changes in the vessels of the organoids include thickening of basement membrane (increased collagen type IV, fibronectin, laminin, perlecan), reduced vessel growth and endothelial/pericyte death. In an animal model, diabetes may also be induced by selecting an animal with organic causes of diabetes, such as a deficiency of pancreatic beta-cells; or by causing pancreatic beta-cell insufficiency. Beta-cell insufficiency may be caused by autoimmune destruction, such as in diabetes type 1, or by chemical toxicity, e.g. induced by streptozotocin.

The prevalence of autoimmune type 1 and especially of type 2 diabetes mellitus (T2D) is increasing constantly, resulting in a global epidemic of already more than 420 million patients. There are various risk factors for T2D, such as obesity, aging, nutritional states and physical inactivity, in addition to genetic pre-dispositions in different populations. The consequences of high blood glucose include damaged blood vessels, leading to arteriosclerosis and chronic diabetic microangiopathies. The structural hallmark of diabetic microangiopathy is the thickening of the capillary basement membranes due to increased expression and deposition of extracellular matrix proteins, in particular of type IV collagen. These changes lead to occlusive angiopathy, altered vascular permeability, or tissue hypoxia, resulting in complications such as heart disease, strokes, kidney disease, blindness, impaired wound healing, chronic skin ulcers, or amputations. Such conditions can be studies in the inventive organoid, e.g. but not necessarily in an animal model.

Although diabetic microvascular changes can occur in dogs, hamsters, or monkeys, no single experimental animal model displays all the clinical features of the vascular changes seen in human patients. Moreover, these vascular changes are insufficiently recapitulated in previous human in vitro cell culture models. Thus, a comprehensive understanding of the vascular changes affecting hundreds of millions of diabetes patients, which cause life-altering morbidities and ever increasing mortalities, is still lacking. The inventive 3D human blood vessel organoids that exhibit morphological features and molecular signatures of bona fide human microvasculature. These human 3D blood vessels can grow vascular trees in vivo in non-human animals, like mice. Importantly, these organoids can be used to model diabetic microangiopathy and to screen for pathways that could be targeted to protect from “diabetes”-induced vascular damage.

The invention further relates to a method of screening a candidate chemical compound for influencing a pathogenesis or a pathology comprising administering said candidate chemical compound to a culture or non-human animal model or during generation of said culture or non-human animal model according to any aspect and embodiment of the invention and monitoring for physiological differences in said culture or animal model as compared to said culture or animal model without administration of the candidate chemical compound. The stem cells used in the method, organoid or non-human animal model used for screening may have or is developing the pathology or is subject to pathogenesis as mentioned above. A method of testing or screening a candidate compound for influencing properties, modification and development of vascular capillaries and their networks is provided, said method comprises contacting cells or a organoid or the animal in a method of any one of the invention with the candidate compound or contacting an organoid of the invention with the candidate compound and maintaining said contacted organoid in culture or in vivo, and observing any changes, such as developmental changes, in the capillaries of the organoid as compared to said organoid without contacting by said candidate compound, including changes (like physiological changes or gene expression changes) in developed or developing capillaries of the organoid.

The contacting step is a treating step of cells to be developed into the inventive organoid or of the organoid or its precursor cell aggregates. The candidate compound may be a small organic molecule, such as molecules with a mass of 100 Da to 5000 Da. Other candidate compounds may be biomolecules such as proteins, nucleic acids or carbohydrates. Further candidate compounds may be bulk chemicals such as solvents, like ethanol—of course used in concentrations generally viable for cells—or polymers. The treatment should be in a concentration wherein a specific effect of the compound can be expected. Various concentrations may be tested in parallel. Usually concentration of candidate compounds is 1 ng/ml to 100 mg/ml, e.g. of 100 ng/ml to 1 mg/ml.

Also provided is a method of screening or testing a candidate therapeutic agent suitable for treating a pathology in the organoid of interest, comprising providing an organoid of the invention, e.g. by performing the inventive differentiation method and administering the candidate agent to said cells at any stage during the method (as above), preferably at all stages, or to the pathology-affected organoid. As above, a change in the vascular network of the organoid is observed as compared to without such candidate agent. Such a change may be e.g. in the thickness of the basal membrane, as e.g. observed in case of diabetes.

This method has been employed in the diabetes model mentioned above. Accordingly, the Notch3 activation pathway, in particular gamma-secretase and its pathways was identified as an ameliorating agent suitable for the treatment of diabetes. Accordingly, the invention also provides the use of a Notch3 activation pathway inhibitor (such as a gamma-secretase inhibitor, a Notch3 inhibitor, DLL4 inhibitor or a combination thereof) in the treatment or prevention of a thickened capillary basement membrane, such as in diabetic vasculopathy, occlusive angiopathy, altered vascular permeability, tissue hypoxia, heart disease, stroke, kidney disease, blindness, impaired wound healing or chronic skin ulcers.

Example Notch3 activation pathway inhibitors, in particular inhibitors of gamma-secretase, Notch3, or DLL4, are inhibitory antibodies and binding partners of gamma-secretase, Notch3, or DLL4. An antibody includes any functional equivalents and derivatives thereof, including antibody fragments such as Fab, F(ab)2, Fv, single chain antibodies (scAb), nanobodies or like camelid antibodies, or an antibody anti gen binding domain. Antibodies specifically binding gamma-secretase, Notch3, or DLL4, are encompassed by the invention. The antibodies may be produced by immunization with full-length protein, soluble forms of the protein, or a fragment thereof. The antibodies of the invention may be polyclonal or monoclonal, or may be recombinant antibodies, such as chimeric antibodies wherein the murine constant regions on light and heavy chains are replaced by human sequences, or CDR-grafted antibodies wherein only the complementary determining regions are of murine origin. Antibodies of the invention may also be human antibodies prepared, for example, by immunization of transgenic animals capable of producing human antibodies (WO 93/12227). The antibodies are useful for detecting gamma-secretase, Notch3, or DLL4 in biological samples, thereby allowing the identification of cells or tissues which produce the protein in addition, antibodies which bind to gamma-secretase, Notch3, or DLL4 (and block inter-action with other binding compounds) have therapeutic use as gamma-secretase, Notch3, or DLL4 inhibitor. Particularly preferred are anti-gamma-secretase antibodies. A preferred anti-Notch3-antibody is tarextumab. Abcam anti-DLL4 antibodies are for example ab7280, ab176876, ab183532.

Further inhibitors of these components may be any (physiological) binding partner, such as receptors or ligands, that sequester gamma-secretase, Notch3, or DLL4 and thus reduce biological activity. Binding partners are preferably binding proteins. An example ligand for Notch 3 is recombinant soluble DLL4 protein. Such a binding partner (which is preferably not cross-linked to a substrate or membrane, e.g. crosslinked on a plate) will bind to the corresponding Notch receptor without activation. Thus recombinant DLL4 protein can act as an inhibtitor since it is not presenteted on a cell surface, such as an endothel cell surface (Scehnet et al. Blood 2007 109(11): 4753-4760; Noguera-Troise et al. Nature 2006 444(7122): 1032-1037). Prefearbly an such binding protein is provided in soluble form, without being immobilized on a solid durface or on a cell membrane, especially also not in complex with another protein. Suc soluble forms bind to the respective target (such as gamma-secretase, Notch3, or DLL4) but fail to activate the signalling cascade but instead inhibit it by blocking the target. Binding proteins may also be provided as sequestering or masking agents that bind the target and mask its effect due to complex formation that prevents binding of activating signalling molecules.

Further Notch3 activation pathway inhibitors, in particular gamma-secretase, Notch3, or DLL4 inhibitors, are small molecule inhibitors. Small molecules are usually small organic compounds having a size of 5000 Dalton or less, e.g. 2500 Dalton or less, or even 1000 Dalton or less. A small molecule inhibitor inhibits the activity of gamma-secretase, Notch3, or DLL4 on diabetic organoids as can be easily tested by the methods disclosed herein. Example gamma-secretase inhibitors are Semagacestat, Avagacestat, R04929097, DAPT, LY3039478 (Crenigacestat), LY411575, Dehydroxy-LY411575, LY 450139, MK-0752, IMR-1, Dibenzazepine, PF-03084014 (Nirogacestat), L-685,458, FLI-06, NGP 555, Flurbiprofen and Sulindac.

Further inhibitors are inhibitory nucleic acids, like siRNA, shRNA or sgRNA (in combination with CRISPR-Cas). RNA interference (RNAi) is a mechanism to suppress gene expression in a sequence specific manner. RNA interference (RNAi) is highly effective methodology for suppression of specific gene function in eukaryotic cells. When applied to cells and organisms, RNAi entails the degradation of target mRNA upon transfection of short interfering RNA (siRNA) oligos or short-hairpin RNA (shRNA) encoding vectors. Various methods of RNAi have been described and are generally known for the altering gene expression in plant cells, drosophila and human melanoma cells as is described for example in US 2002/0162126 and US 2002/0173478. The siRNA for use in the methods and compositions of the invention are selected to target a desired molecule of the gamma-secretase, Notch3, or DLL4 signalling pathway or combinations of such molecules. In this manner they are targeted to various RNAs corresponding to a target gene. It is understood by one of skill in the art that the siRNA as herein described may also include altered siRNA that is a hybrid DNA/RNA construct or any equivalent thereof, double-stranded RNA, microRNA (miRNA), as well as siRNA forms such as siRNA duplications, small hairpin RNA (shRNA) in viral and non-viral vectors and siRNA or shRNA in carriers. There exists several methods in the art for inhibiting gene expression using RNAi such as described for example in WO 02/055692, WO 02/055693, EP 1 144 623 B1 and WO 03/074654. By using a siRNA therapy any cellular factor can be targeted and inhibited for the inventive gamma-secretase, Notch3, or DLL4 antagonizing and inhibiting therapy. Therefore, any such compound can be used as a gamma-secretase, Notch3, or DLL4 inhibitor.

Also provided are inhibitors in form of encoding nucleic acids. The inhibitory nucleic acid, antibody or binding partner (e.g. receptor or ligand) may be encoded on nucleic acid that expresses said inhibitors in a cell, thereby exhibiting the inhibitory action.

The inhibitor is usually administered in a therapeutically effective amount, an amount that reduces gamma-secretase, Notch3, or DLL4 activity to significantly decrease diabetic morphology. Preferably the gamma-secretase, Notch3, or DLL4 activity is reduced by at least 25%, by at least 30%, by at least 40%, by at least 50%, by at least 60%, by at least 70% by at least 80% or by at least 90% as compared to amounts without treatment (but otherwise similar conditions). In preferred embodiments this reduction equates to gamma-secretase, Notch3, or DLL4 intracellular levels.

The inhibitor may be provided in a pharmaceutical composition. Pharmaceutical compositions or formulations for therapeutic or prophylactic use may comprise a pharmaceutically acceptable diluent, carrier, solubilizer, emulsifier, preservative and/or adjuvant. The invention also provides for pharmaceutical compositions comprising a therapeutically effective amount of a gamma-secretase, Notch3, or DLL4 inhibitor. The term “therapeutically effective amount” means an amount which provides a therapeutic effect for a specified condition and route of administration. The composition may be in a liquid or lyophilized form and comprises a diluent (Tris, acetate or phosphate buffers) having various pH values and ionic strengths, solubilizer such as Tween or Polysorbate, carriers such as human serum albumin or gelatin, preservatives such as thimerosal or benzyl alcohol, and antioxidants such as ascorbic acid or sodium metabisulfite. Selection of a particular composition will depend upon a number of factors, including the condition being treated, the route of administration and the pharmacokinetic parameters desired. siRNA formulations are preferably administered in liposome formulations.

The invention further provides the use of an artificial blood vessel organoid according to the invention, such as obtained from the invetive culture, preferably with a hydrogel with collagen, as an implant in a tissue replacement therapy. A therapy of using the invetive organoid may comprise placing the artificial blood vessel organoid into a wound and letting said artificial blood vessel organoid culture integrate into the wound. The use as an implant can comprise placing the organoid into a subject to be treated, in particular at a position that requires connective tissue regrowth. Sich regrowth can be stalled, e.g. due to diseases that impair regrowth, like diabetes, or medication or other therapies, such as in a chemotherapoy or radiation, e.g. as in a chermotherapy or radiation accidents. A wound is preferably treated. Such a wound may be a chronic wound, in particular a wound that fails to close in 30 days or 60 days or even 90 days. A chronic wound can be due to the above circumstances, diseases, medication or therapy. In particular, the wound is a diabetic wound, such as a diabetic foot ulcer, or a burn, such as a third degree burn. The wound may comprise a skin wound. A skin wound may comprise damage to both epidermal and dermal layers. Any wound ,including a skin wound may comprise trauma to the underlying muscles, bones, and tendons. The therapy may comprise wound cleaning, in particular removal of dead tissue to ease regrowth.

In such a therapy, one or more organoids, the number depending on the size of the wound, are placed into the volume to be treated, such as a wound, and the organoids are allowed to integrate with the tissue surrounding the volume. Said volume is preferably surrounded by tissue of the patient in at least 50%, preferably at least 75%, of the one or more organoids surface area facing the outside of said one or more organoids (not counting internal surface area in case of more than one organoids that face other organoids). This means that the volume is mostly internal and able to integrate within the subject. Woundas may be internal or open wounds. Even open woundas may have such a volume facing an open surface, such as a skin wound.

Letting the organoid integrate into the wound usually comprises regenerative processes that are improved by the prsesnce of the blood vessels of the inventive organoid. Said blood vessels are able to connect with the circulatory system of the patient and improve oxygenation of damaged tissue and by concequence its regeneration.

In other to avoid immunereactions against the organoid and its cells, the cells of the organoid are preferably of the same organism as the patient (preferably both human, or both of the same non-human animanl, preferably mammal) and are a MHC match to the patient. To quickly provide such a fitting organoid, it is possible to create a organoid library with various documented MHC-types. Its organoids can be quickly provided to a patient.

Further provided is a kit of compounds and substances. The kit may comprise means to perform any of the inventive methods. Of course, not all substances need to be included since some are standard chemicals or usually available. Nevertheless, preferably the core substances are provided. In other kits, rarer substances are provided. The inventive kits or their substances may be combined. Components in the kit are usually provided in separate containers, such as vials or flasks. Containers may be packaged together. Preferably the kit comprises a manual or instructions for performing the inventive methods or their steps.

Provided is a kit suitable for the generation of an artificial blood vessel organoid according to any inventive method. The kit may comprise (i) a Wnt agonist or a GSK inhibitor; (ii) a vascular differentiation factor selected from VEGF, preferably VEGF-A, a FGF, preferably FGF-2, a BMP, preferably BMP4; (iii) a collagenous 3D matrix, preferably comprising 10%-50% laminin, 20%-70% collagen I, and/or 2%-30% collagen IV (all wt.-%).

Preferably, the kit comprises a 3D matrix as described above, or their components to generate such a 3D matrix. Matrix components may be provided in solid state, such as lyophilized state to be reconstituted to the matrix, e.g. by hydration. Any matrix or their components as described above may be included in the kit. Preferably the matrix is a hydrogel, especially a collagenous hydrogel as described above. The kit may comprise such reconstitutable (preferably collagenous) components. Further preferred matrix components are carbohydrates, in particular in polymeric form (polysaccharides). A preferred polysaccharide is agarose.

Any kit may further comprise cell growth nutrients, preferably DMEM/F12, knock-out serum replacement (KOSR) medium, Glutamax, or essential amino acids and/or non-essential amino acids (NEAA), or any combination thereof. Any compound mentioned in the examples can be included in the kit.

It is contemplated that any method or product described herein can be implemented with respect to any other method or product described herein and that different embodiments may be combined.

The kit may further comprise instructions for performing the inventive method. Such instructions may be in printed form or in a computer-readable format on a suitable data carrier.

The claims originally filed are contemplated to cover claims that are multiply dependent on any filed claim or combination of filed claims. It is contemplated that any embodiment discussed herein can be implemented with respect to any method or product of the invention, and vice versa. Any embodiment discussed with respect to a particular condition can be applied or implemented with respect to a different condition. Furthermore, compositions and kits of the invention can be used to achieve methods of the invention.

“Comprising” is understood as an open-ended term, i.e. allowing further components or steps of substance. “Consisting of” is understood of a closed term without any further components or steps of substance.

Throughout this application, the term “about” may be used to indicate that a value includes the standard deviation of error for the device or method being employed to determine the value or in a set value may refer to ±10%.

The present invention is further defined in the following preferred embodiments and definitions, that are all combinable with the above detailed description:
1. A method of generating an artificial blood vessel organoid, comprising providing stem cells capable of vascular differentiation, stimulating mesoderm differentiation in said stem cells, stimulating vascular differentiation in said stem cells, developing a cell aggregate from said stem cells, embedding said cell aggregate in a collagenous 3D matrix and stimulating vascular differentiation of the aggregate in said collagenous 3D matrix.
2. The method of 1, wherein the collagenous 3D matrix comprises at least 50 wt.-% collagen.
3. The method of 1 or 2, wherein the collagenous 3D matrix comprises 10%-50% laminin, 20%-70% collagen I, and/or 2%-30% collagen IV; preferably further 0.5%-10% nidogen, 0.5%-10% heparan sulfate proteoglycan, and/or 0.5%-10% entactin (all wt.-%).
4. A method of generating an artificial blood vessel organoid, comprising embedding vascular stem cells in a collagenous 3D matrix comprising 10%-50% laminin, 20%-70% collagen I, and/or 2%-30% collagen IV and stimulating vascular differentiation of said stem cells in said collagenous 3D matrix (all wt.-%).
5. The method of 4, wherein the vascular stem cells are generated by differentiating mesodermal stem cells into vascular stem cells, preferably wherein the mesodermal stem cells have been obtained by stimulating mesodermal differentiation in pluripotent stem cells.
6. The method of any one of 1 to 5, wherein the stem cells cagable of vascular differentiation are pluripotent stem cells, preferably induced pluripotent stem cells.
7. The method of any one of 1 to 6, wherein the cell aggregate that is embedded in a collagenous matrix comprises at least 50 cells.
8. The method of any one of 1 to 7, wherein the mesoderm differentiation comprises treating the stem cells with a Wnt agonist or a GSK inhibitor, preferably CHIR99021.
9. The method of any one of 1 to 8, wherein vascular differentiation in said stem cells comprises treating the stem cells with a VEGF, preferably VEGF-A, and/or a FGF, preferably FGF-2, and/or a BMP, preferably BMP4, and/or low oxygen conditions of 12% (v/v) or less atmospheric oxygen.
10. The method of any one of 1 to 9, wherein vascular differentiation of the aggregate comprises treating cells of the aggregate with a VEGF, preferably VEGF-A, and/or a FGF, preferably FGF-2.
11. The method of any one of 1 to 10, wherein said aggregate is embedded in the collagenous 3D matrix at day 7 to 15 from the start of aggregate formation.
12. The method of any one of 1 to 11, wherein the cells of the aggregate are cultured in said 3D matrix for at least 5 days, preferably for at least 7 days.
13. The method of any one of 1 to 12, wherein the 3D matrix is a hydrogel, preferably having a viscoelastic storage modulus G′ of 10 to 30.
14. An artificial blood vessel organoid culture comprising an interconnected network of vascular capillaries, said capillaries comprising endothelium and a basal membrane with perivascular pericytes, wherein said organoid is produced by a method of any one of 1 to 13 and/or wherein the capillaries are embedded in an artificial 3D matrix comprising a hydrogel with collagen and/or wherein the organoid culture comprises 40 to 1000 blood vessels as counted by counting individual vessels and vessels between capillary intersections.
15. The artificial blood vessel organoid culture of 14, wherein the vascular capillaries have an average diameter of from 1 μm to 30 μm.
16. The artificial blood vessel organoid culture of 14 or 15, wherein the ratio of endothelial cells to perivascular pericytes is between 100:1 to 1:5.
17. The artificial blood vessel organoid culture of any one of 14 to 16, wherein the vascular capillaries comprise mature endothelial cells and/or mature pericytes.
18. A method of providing a non-human animal model with human vascular capillaries, wherein said human capillaries comprise endothelium and a basal membrane with perivascular pericytes, comprising the steps of introducing a human blood vessel organoid of any one of 14 to 17 into a non-human animal and letting said organoid grow its vascular capillaries, preferably, wherein said human organoid is introduced onto or into the kidney of the non-human animal.
19. A non-human animal model comprising an inserted artificial blood vessel organoid culture according to any one of 14 to 18.
20. A non-human animal model with human vascular capillaries, wherein said human capillaries comprise endothelium and a basal membrane with perivascular pericytes.
21. The non-human animal model of 19 or 20, wherein the vascular capillaries of the artificial blood vessel organoid culture or the human vascular capillaries are perfused by the blood circulatory system of the non-human animal.
22. The method or culture or non-human animal model of any one of 1 to 21, wherein the blood vessels or capillaries are subjected to pathogenesis and said organoid or human animal model is a model of pathology.
23. The method or culture or non-human animal model of 22, wherein pathogenesis comprises hyperglycaemia and/or inflammation and/or wherein said pathology is diabetes, preferably wherein said inflammation comprises exposure to one or more inflammatory cytokines, preferably TNF-alpha and/or IL-6.
24. The method of screening a candidate chemical compound for influencing a pathogenesis or a pathology comprising administering said candidate chemical compound to a culture or non-human animal model or during generation of said culture or non-human animal model according to any one of 1 to 23 and monitoring for physiological differences in said culture or animal model as compared to said culture or animal model without administration of the candidate chemical compound.
25. The method of investigating a developmental vascular tissue effect, e.g. a defect, in particular a developmental defect, comprising (i) decreasing or increasing the expression in a gene of interest in a cell at any stage during the method or to the organoid or animal of any one of claims 1 to 21, or (ii) administering a candidate chemical compound of interest to a cell during development of the organoid at any stage during the organoid or animal of any one of claims 1 to 21.
26. Use of an artificial blood vessel organoid according to any one of claims 14 to 17 as an implant in a tissue replacement therapy, especially preferred a therapy comprising placing the artificial blood vessel organoid into a wound and letting said artificial blood vessel organoid culture integrate into the wound.
27. Use of a Notch3 activation pathway inhibitor (such as a gamma-secretase inhibitor, a Notch3 inhibitor, DLL4 inhibitor or a combination thereof) in the treatment or prevention of a thickened capillary basement membrane, such as in diabetic vasculopathy, occlusive angiopathy, altered vascular permeability, tissue hypoxia, heart disease, stroke, kidney disease, blindness, impaired wound healing or chronic skin ulcers.
28. A kit suitable for the generation of an artificial blood vessel organoid according to any method of 1 to 13, comprising (i) a Wnt agonist or a GSK inhibitor; (ii) a vascular differentiation factor selected from VEGF, preferably VEGF-A, a FGF, preferably FGF-2, a BMP, preferably BMP4; (iii) a collagenous 3D matrix, preferably comprising 10%-50% laminin, 20%-70% collagen I, and/or 2%-30% collagen IV (all wt.-%).

The present invention is further exemplified by the following figures and examples, without being limited to these particular embodiments of the invention.

### FIGURES

FIG. 1. Generation of human vascular networks from human stem cells. a, Schematic of the protocol to differentiate human embryonic stem cells (ESCs) and human iPSCs into vascular networks and free-floating vascular organoids. Bottom panels show representative morphologies observed at the indicated steps of differentiation. b,c, Immunofluorescence for CD31-expressing endothelial cells shows the establishment of complex, interconnected vascular networks in the Collagen I/Matrigel matrix. d, 3D-reconstruction of the CD31⁺ vascular network, based on confocal imaging. The scales of reconstruction are indicated in the 3 axes. e, TNFα-mediated activation of 3D endothelial networks revealed by the induction of ICAM-1 expression. f-h Endothelial (green) and pericyte (red) coverage of vascular networks determined by CD31⁺ endothelium and the pericyte-specific markers CNN1, PDGFRβ, and SMA. Formation of the basement membrane is shown by Collagen type IV (ColIV) expression. i, Self-organizing human capillary organoids shown by immunofluorescence for Collagen Type IV (ColIV) to visualize the deposition of a basement membrane that coats the endothelial tubes. Of note, data in b-i are from blood vessels organoids derived from human embryonic stem cells. DAPI staining is shown to image nuclei. Magnifications are indicated in each panel.

FIG. 2. Generation of mature, continuous human capillaries. a, Free-floating organoids show a dense endothelial network (CD31⁺) tightly covered by pericytes, as determined by PDGFRβ expression. A 3D-reconstruction is also shown for the entire free-floating organoid (top left panel). b, Endothelial lumen formation in free-floating vascular organoids shown by immunofluorescence to CD31⁺ endothelium and H&E staining. c, Representative electron microcopy of free-floating vascular organoids. Note the generation of lumenized, continuous capillary-like structures with the appearance of tight junctions (white arrowheads) and a basement membrane (black arrows). L, lumen; E, endothelial cell. d, CD31⁺ tip cells (arrowheads) mark newly forming cells. Note the absence of a ColIV⁺ basement membrane at the site of angiogenesis. Magnifications are indicated in each panel.

FIG. 3. Establishment of a functional human vascular tree in mice. a, Transplantation of human vascular organoids into the kidney capsule of NOD/SCID mice. Top left panel indicates site of transplantation (arrow). The human organoid derived vasculature is visualized by a human-specific CD31 antibody that does not cross react with murine endothelium, exemplified by staining of mouse kidney (inset). b-c, Functional human vasculature (detected by human-specific anti-CD31 immunostaining, hCD31, red) in mice revealed by FITC-Dextran perfusion (green). d, Infusion of the human specific anti-CD31 antibody to label the perfused human blood vessels. Murine vessels are visualized by a mouse-specific anti-CD31 antibody (mCD31, green). e, Representative arteriole (A) and venule (V) appearing within the human vascular organoid transplants shown by H&E stained histological sections. f, Generation of human arterioles (A) and venules (V) in the human transplanted blood vessel organoids. Arterioles are shown by staining for human CD31⁺ endothelial cells (red), tightly covered with vascular smooth muscle cells (vSMC) detected by SMA, Calponin and MYH11 immunostaining. Venules show a typical flat endothelial phenotype and sparse vSMC coverage. Endothelial cells of murine arterioles do not cross react with the human specific CD31 antibody, as shown for kidney blood vessels (bottom right panel). Magnifications are indicated in each panel. g, Representative axial T2-weighted image, blood flow (perfusion), relative blood volume (rBV), mean transit time (MTT) and leakage (K₂) measured by MRI. The axial plane was chosen so that both kidneys (outlined in white) and the implant (outlined in red) are visible. Muscle tissue is outlined in green. The quantitative values (+/−SD) for perfusion, rBV, MTT and K₂ are given in the table below. n=3 mice analysed.

FIG. 4. Modelling diabetic microvasculopathy in human blood vessel organoids. a, Basement membrane thickening of dermal capillaries in skin biopsies of a late-stage type 2 diabetic patient shown by PAS staining (left) and staining for CD31⁺ endothelial cells and ColI V to detect the basement membrane. Dermal blood vessels from a non-diabetic patient are shown as control. b, Representative electron microscopy of dermal capillaries of a late-stage type 2 diabetic and a non-diabetic patient shows the formation of an abnormally thick basement membrane in diabetic patients (two-sided arrows) as compared to basal membrane in non-diabetic controls (arrowheads). L, lumen; E, endothelial cell; P, pericyte. Bar graph shows quantification of basement membrane thickening (mean values+/−SD). n=6. *** p<0.001 (unpaired, two-tailed t-test). c-d, Human blood vessel organoids show increased Collagen type IV deposition upon hyperglycemia [75 mM Glucose], which is significantly further increased by treatment with high glucose in combination with the proinflammatory cytokines IL6 [1 ng/mL] and TNFα [1 ng/ml] (“diabetic cocktail”). c, Representative images of basement thickening; insets indicate confocal cross-sections of luminal vessel. d, Confocal cross-sections were used to quantify collagen type IV thickening; individually measured vessels are shown as dots. >130 vascular lumens were analysed for each experimental condition from 3 independent biological replicates. ***p<0.001 (Student's t-test). e, High glucose/IL6/TNFα treatment leads to a marked expansion of the Col IV-positive basement membrane lining the human capillaries. Right panels show 3D reconstructions of basement membrane thickening directly coating the CD31⁺ endothelial tubes. Caplonin immunostaining marks pericytes. f, Representative electron microscopy images of vascular organoids, cultured under “diabetic” and non-diabetic condition, confirm the marked basement membrane thickening upon diabetic treatment. Note multiple layers of basement membrane in the diabetic condition (two-sided arrows) which cannot be observed in the control organoids (arrowheads). L, lumen; E, endothelial cell; P, pericyte. All magnifications are indicated.

FIG. 5. Inhibition of y-secretase abrogates vascular basement membrane thickening in diabetic blood vessel organoids. a, Transcriptome analysis of CD31⁺ endothelial cells FACS sorted from vascular organoids cultured under diabetic (high glucose/IL6/TNFα) and non-diabetic conditions. Heat maps of differentially expressed genes and the top 5 upregulated genes (ranked by p-value) and GO:biological processes of upregulated genes are shown comparing diabetic vs non-diabetic conditions. Common GO:Molecular function terms comparing upregulated genes from “diabetic” blood vessel organoids and type II patient-derived dermal endothelial CD31⁺ cells are plotted with their respective p-value. Upregulated genes in patients were derived from sorted CD31⁺ endothelial cells from type II diabetes patients compared to those from non-diabetic individuals. b,c, Commonly prescribed diabetic drugs do not affect basement membrane thickening upon treatment of human blood vessel organoids with the diabetic cocktail (high glucose/IL6/TNFα). b, Representative images of basement membrane thickening using Collagen IV (ColIV) staining. Insets indicate confocal cross-sections of luminal vessels covered by Collagen IV (green). c, Optical cross-sections were used to quantify basement membrane thickening. Each lumenized vessel is shows as a dot. >130 lumens were analysed for each experimental condition from 3 independent biological replicates. *** p<0.001 (Student's t-test comparing vehicle and drug treated organoids with organoids cultured in parallel under non-diabetic conditions). Besides the indicated comparison, all other drug treatments were p<0.001 as compared to the non-diabetic condition and not significant as compared to their respective vehicle-diabetic condition control. Drug doses and cultured conditions are described in the methods. d,e, Inhibition of y-secretase by DAPT abrogates thickening of the vascular basement membrane in blood vessel organoids cultured under “diabetic” conditions, visualized by ColIV. d, Representative images of basement thickening in diabetic blood vessels treated with small molecule inhibitors of various signaling pathways. Insets show confocal cross-sections of luminal vessels enveloped by Collagen IV (ColIV, green). e, Confocal cross-sections were used to quantify basement membrane thickening. >130 lumenized vessels (each vessel is show as individual dot) were analysed for each experimental condition from 3 independent biological replicates. ** p<0.01; *** p<0.001 (Student's t-test). Of note, besides the indicated comparisons, all other drug treatments were p<0.001 as compared to the non-diabetic condition and not significant as compared to the vehicle-diabetic condition control. Drug doses and cultured conditions are described in the methods. f,g, Prevention of basement membrane thickening by the y-secretase inhibitor DAPT is dose dependent. Quantification shows ColIV thickness of >130 lumen lumenized structures (dots) from at least different organoids exposed to the indicated conditions. ***p<0.001 (Student's t-rest).

FIG. 6. Differentiation of human ES cells into vascular organoids. a, Co-expression of endothelial markers CD31 and VE-Cadherin in 3D endothelial tubes. Representative data are shown from ESC-derived organoids grown in a collagen I matrix. b,c Human iPS cells efficiently differentiate in CD31⁺ positive tubes. b, shows representative images of experiments repeated more than 20 times c, 3D-reconstruction of the CD31⁺ vascular network derived from iPS cells, based on confocal imaging. The scales of reconstruction are indicated in the 3 axes. Data in b and c and from organoids grown in the collagen matrix. d, 3D-reconstruction is shown for an entire free floating organoid derived from iPS cells. Data were derived using confocal imaging of the entire organoid imaged with anti-CD31 antibodies. The scales of reconstruction are indicated in the 3 axes. All magnifications are indicated in the panels.

FIG. 7. Molecular characterization of vascular organoids. a, Heatmap of the transcriptome (RNAseq) of FACS sorted CD31⁺ endothelial cells from vascular organoids compared to genotype-tissue expression (GTEx) RNASeq data from the indicated tissues. The gene expression profiles of in vitro generated endothelial tubes clusters most closely with human blood vessel tissues (GTEX_Artery_Coronary, GTEX_Artery_Aorta GTEX_Artery_Tibial) b, Heatmap of marker genes for pluripotency, perivascular cells and endothelial cells. FACS sorted CD31⁺ endothelial cells from vascular organoids were compared to previously published primary and differentiated endothelial cells derived from iPS cells in 2D culture conditions (Patsch et al. Nat. Cell Biol. 17, 994-1003 (2015).). c, Laminin expression (blue) to image the basal membrane encircling the CD31⁺ endothelial tubes (green) in vascular organoids. A representative image is shown for a human ESC-derived vascular organoid, grown in a collagen I matrix. The Magnification is indicated in the panel.

FIG. 8. Common diabetic rodent models do not display a basement membrane thickening in the skin microvasculature. a, Quantification of the basement membrane thickness of dermal blood capillaries in the indicated rat and mouse models of diabetes as compared to their non-diabetic control cohorts. See Supplementary Table 2 for details. Data are shown as mean values+/−SD of analyzed blood vessels. n>5 animals per cohort. Age matched C57 BL/KsJ and C57 BL/Ks WT mice were used as controls. For the ZDF rat model, heterozygous rats (fa/+) were used as controls. Basement membrane thickening was determined based on morphometric analyses of Collagen IV immunostaining. b, Representative images of skin sections of various mouse models to demonstrate ColIV (green) deposition covering CD31⁺ (red) positive blood vessels.

FIG. 9. Basement membrane thickening in human ESC-derived vascular organoids. a, ES-cell derived vasculature was treated with diabetic media (high glucose/IL6/TNFα) or cultured under normal conditions (non-diabetic). Thickening of the basement membrane was visualized with a Col IV specific antibody (green) around CD31 positive (red) endothelial tubes. Insets show confocal cross-sections. CNN1 marks pericytes. b, Enhanced Col IV expression in diabetic vascular organoids. Col4a1 and Col4a2 expression was determined via qPCR in vascular organoids that have been treated with diabetic media (high glucose/IL6/TNFα) for 2 weeks and compared to non-treated control organoids. Values are shown as means+/−SD.* p<0.05 (Student's t-rest). A pool of >15 vascular organoids was used in 2 independent experiments.

FIG. 10. Efficient differentiation of pluripotent stem cells into endothelial cells and pericytes in vascular organoids. a, Established vascular networks at day 18 and late vascular organoids at day 30 were analyzed by FACS for their endothelial cell and pericyte content. In both cases, >80% of the cellular populations after differentiation exists of endothelial cells (CD31⁺) and pericytes (CD140b⁺). P, pericytes; EC, endothelial cell b, Only little (˜1%) hematopoietic cells (CD45⁺) are generated and around 10% of the cells found after differentiation are mesenchymal stem cell like cells (CD73⁺, CD90⁺).

FIG. 11. Mature endothelial cells in vascular organoids. a, Endothelial cells in vascular organoids express von Willebrand factor (vWF) and show formation of Weibel-Palade bodies (right panel). b, Binding of Ulex Europaeus Agglutinin 1 (UEA-1) to capillary structures in vascular organoids indicates the presence of mature endothelial cells. c, Mature endothelial cells in free floating vascular organoids efficiently take up acetylated LDL (ac-LDL).

FIG. 12. Basement membrane thickening of transplanted human vessels in diabetic mice. Vascular organoids were transplanted into immuno-compromised NOD/SCID/Gamma (NSG) mice that were subsequently (1 month later) treated with Streptozotocin (STZ) to induce severe hyperglycemia. After 3 months, the human transplants were harvested and analyzed for diabetic basement membrane thickening. Transplanted organoid-derived blood vessels were identified using a human specific CD31 (hCD31) antibody. Severe basement membrane thickening in human vascular organoids derived from diabetic mice (STZ) compared to normoglycemic mice (Ctrl) shown by Collagen type IV staining (upper panel) and electron microscopy (middle panel; arrows denote deposited collagenous fibrils). In contrast, the endogenous blood vessels of the murine kidney do not show changes of the basement membrane (lower panel; A, arteriole, C capillary) at that stage. Absence of CD31 signal in the kidney demonstrates human specificity of the antibody.

FIG. 13. Diabetic vessel regression is recapitulated in human vascular organoid transplants. Human vascular organoids were transplanted into NSG mice that were after 1 month treated with STZ to induce diabetes. Transplants from diabetic mice (STZ) show an overall reduced vascular density compared to normoglycemic mice (Ctrl) shown by endothelial (CD31) and pericyte (SMA) staining (upper panel) 3 month after diabetes induction. Human blood vessels in diabetic mice (STZ) show signs of vessel regression such as endothelial apoptosis indicated by rounded up cells (filled arrowheads) and the lack of endothelial cells at SMA positive vessel walls (empty arrowheads).

FIG. 14. Blocking Notch3 receptor or the ligand D114 inhibits diabetic basement membrane thickening. Vascular Organoids were treated in vitro for 2 weeks with diabetic media (hyperglycemia+IL-6 +TNF-a) which leads to massive basement membrane thickening of the capillaries as compared to control media (Ctrl) shown by Collagen type IV staining (Col IV). To unravel the direct targets of the previously identified y-secretase inhibitor DAPT, which can prevent diabetic vascular basement membrane thickening, specific members of the Notch pathway were inhibited using functional blocking antibodies (α-Notch1, α-Notch3, α-Jagged1) and non-crosslinked recombinant proteins (D111, D114) under diabetic conditions. Whereas blocking Notch-1, Jagged-1 or D111 did not impact on the diabetes mediated thickening of the vascular basement membrane, blocking Notch-3 or D114 completely blocked basement membrane thickening. In the vascular system, the Notch3 receptor is specifically expressed on pericytes and the Notch ligand D114 on the endothelium, which suggests now that the crosstalk between these two cell types via Notch3/D114 mediates the diabetic vascular basement membrane thickening.

FIG. 15. Cellular and functional characterization of vascular organoids. a, FACS analysis to determine different cell populations present in initially generated vascular networks and latestage vascular organoids (NC8). The percentages of CD31⁺ endothelial cell, PDGFR-β⁺ pericytes, CD45⁺ haematopoietic cells, and CD90⁺CD73⁺ mesenchymal stem cell (MSC)-like cells. Bar graphs in the right panels indicate the relative populations of endothelial cells (ECs) and pericytes (P) in the vascular networks and vascular organoids. Graph represents mean±S.E.M from n=2 independent experiments with >50 vascular networks/organoids per experiment. b, Heatmap of prototypic marker genes for pluripotency, pericytes and endothelial cells. FACS sorted CD31⁺ endothelial cells (EC) and PDGFR-β⁺ pericytes (P) from vascular networks or vascular organoids were analyzed by RNAseq and compared to the parental iPSC line (NC8). c, TNFα-mediated activation of vascular organoids (NC8) revealed by the induction of ICAM-1 expression. ICAM-1 induction was determined 24 hours after addition of TNFα (dose). DAPI was used to counterstain nuclei. d, von Willebrand Factor (vWF) expression in endothelial cells (CD31⁺) from vascular organoids (NC8). Col IV staining is also shown to outline the basal membrane. Right panels show electron microscopy, revealing the appearance of Weibel Palade bodies. e, Endothelial networks (CD31⁺) of vascular organoids (NC8) take up acetylated low-density lipoprotein (ac-LDL) f, Vascular organoids (NC8) stain positive for the lectin Ulex europaeus agglutinin 1 (UEA-1). Scale bars: d=50 μm, 500 nm (EM-upper panel), 100 nm (EM-lower panel), e,f=100 μm or as indicated in the image.

FIG. 16. Analysis of diabetic vascular organoids. a,b FACS analysis of vascular organoids (H9) to determine the percentages of (a) the CD31⁺ endothelial cell fraction and (b) PDGFR-β⁺ pericytes cultured in non-diabetic and diabetic (high glucose/IL6/TNFα) media.

FIG. 17. Inhibition of y-secretase abrogates diabetic microvasculopathy of human blood vessel organoids a, Vascular permeability was assessed by i.v. injection of FITC-Ddextran and costained with hCD31 to visualize the human vasculature. Note the diffuse FITC signal in the diabetic STZ mice which indicates vessel leakage. DAPT treatment normalized diabetic vessel permeability. b, Quantification of vessel leakage determined by FITC-Dextran extravasation. n=(Control=3, STZ=7, STZ+DAPT=5) mice. **p<0.01, *p<0.05 (One-way ANOVA). c,d, DAPT treatment restores human vessel density in diabetic STZ mice. Capillary density of human vascular transplants was determined by staining with human specific anti-CD31 antibodies (black). c, Quantification of human blood vessel density in transplanted vascular organoids. n=(Control=3, STZ=5, STZ+DAPT=4) mice. ***p<0.001 (One-way ANOVA). d, Representative images of human CD31⁺ blood vessel density in control, STZ, and STZ+DAPT treated mice. Scale bars, a,d=50 μm or as indicated in the image.

FIG. 18. Identification of D114-Notch3 as candidate pathway for diabetic vascular basement membrane thickening. a, Representative images of basement membrane stained for Col IV in blood vessel organoids (derived from NC8 iPSCs) exposed to high glucose/IL6/TNFα (diabetic) and treated with antibodies against Jagged-1, Notch1, Notch3, or recombinant D111 and D114. Insets show confocal cross-sections of individual vessels surrounded by collagen type IV (Col IV, green). Thickness of continously surrounded lumina by Col IV was measured in optical cross-sections. For quantifications (right panel), a total of >130 lumens were analysed for each experimental condition from 3 independent biological replicates with equal sample size. Each individual measurement from a lumenized vessel is shown as a dot. Representative images and quantifications for non-diabetic organoids are shown as controls. ***p<0.001 (One-way ANOVA). b, Representative images of basement membranes stained for Col IV from control, D114 KO, and Notch3 KO vascular organoids (NC8 iP-SCs) exposed to high glucose/IL6/TNFα (diabetic) or maintained under standard culture conditions (non-diabetic). Thickness of continously surrounded lumina by Col IV was measured in optical cross-sections. Each individual measurement from a lumenized vessel is shown as a dot in the right panel. A total of >180 lumens were analysed for each experimental condition from 3 independent biological replicates with equal sample size. ***p<0.001 (One-way ANOVA). c, STZ mice transplanted with human vascular organoids (H9 ESCs) were treated with a Notch3 blocking antibody and transplants stained for the basement membrane marker Col IV and human specific CD31 to visualize human blood vessels. Basal membrane thickness of individual human blood vessels (hCD311 was determined based on Col IV staining. n>140 vessels. ***p<0.001 (One-way ANOVA). n=(Control=3, STZ=3, STZ+αNotch3=2) mice. Scale bars, a,b,c=50 μm, c insert=10 μm.

FIG. 19. Creation of D114 and Notch3 knock out iPSCs and expression of Notch receptor/ligands in endothelial cells and pericytes. a, b, CRISPR/Cas9 genome editing was used to generate D114 and Notch3 knock out iPSCs (NC8). Single guide RNAs (sgR-NAs) are indicated in the Notch3/D114 sequence as well as generated indels. c, Western blot shows ablation of Notch3 expression in target iPSCs. Clone #4 (red) was used for functional assays. FL, full length Notch3; TTM, transmembrane Notch3 subunit. d, Immunostaining in vascular organoids shows expression of D114 in endothelial cells (CD31⁺) but not in CRISPR/Cas9 genome edited iPSCs. Scale bar: e=50 μm. e, Heat map of Notch receptors/ligands expressed in endothelial cells (ECs) and pericytes isolated from vascular organoids by FACS sorting. Scale shows log (normalized FKPM).

FIG. 20. Phenotypical characterization of vascular organoids. a, Co-culture of differentiated (NC8) endothelial cells and pericytes in a Collagen 1/Matrigel matrix. The formed endothelial networks (CD31⁺), showed only weak interaction with pericytes (PDGFR-β⁺) and were not enveloped by a Col IV⁺ basement membrane. b, Successful generation of vascular networks from embryonic stem cells (H9) and two independent iPS cell lines. Note how PDGFR-β⁺ pericytes are in close proximity to the endothelial tubes (CD31⁺) and the formation of a Col IV⁺ basement membrane.

FIG. 21. Several y-secretase inhibitors prevent diabetes induced vascular basement membrane thickening in human vascular organoids. Vascular organoids were cultured in diabetic media (75 mM Glucose, 1 ng/mL IL-6, 1 ng/mL TNF-α) in the presence or absence of γ-secretase inhibitors (10 μM RO4929097, 1 μM Dehydroxy-LY411575, 1 μM LY411575). Subsequently, organoids were fixed and stained for endothelial cells (CD31), pericytes (PDGFRβ) and for the vascular basement membrane protein Col IV. Representative images are shown. Diabetic conditions increase the amount of Col IV+basement membrane (vehicle). Treatment with 3 independent γ-secretase inhibitors prevent the increase of basement membrane (Col IV) under diabetic conditions. Scale bar 50 μm.

### EXAMPLES

### Example 1

### Materials & Methods

Human stem cells and differentiation into vascular organoids. All experiments presented were done in either the human iPS cell line NC8 (Pripuzova et al. Stem Cell Res. 14, 323-338 (2015)) or the human embryonic stem cell (ESC) line H9 (Thomson et al. Science 282, 1145-1147 (1998)). All stem cells were cultured under chemically defined, feeder-free conditions as previously described (Chen et al. Nat. Methods 8, 424-9 (2011)). For differentiation, H9 ESCs or NC8 iPS cells were disaggregated using 0.5 mM EDTA for 2 min and subsequently incubated with 0.1% Stempro Accutase (Life Technologies) for 3 min. 2×10⁵ cells were resuspended in differentiation media (DMEM:F12 medium, 20% KOSR, Glutamax, NEAA; all from Gibco) including 50 μM Y-27632 (Calbiochem) and plated into one well of an ultra-low attachment surface 6 well plate (Corning) for cell aggregation. Cell aggregates were treated on day 3 with 12 μM CHIR99021 (Tocris) and on days 5, 7 and 9 BMP4 (30 ng/mL, Stemcell Tech.), VEGF-A (30ng/mL, Peprotech), and FGF-2 (30 ng/mL, Miltenyi) were added. On day 11, cells were switched to media containing VEGF-A (30ng/mL), FGF-2 (30 ng/mL) and SB43152 (10 μM) to balance the endothelial/pericyte ratio. The resulting cell aggregates were embedded on day 13 in Matrigel:Collagen I (1:1) gels and overlaid with differentiation media containing 100 ng/mL VEGF-A and 100 ng/ml FGF-2. This differentiation medium was changed every 2nd to 3rd day. Around day 18 vascular networks were established and either directly analysed or networks from individual cell aggregates were cut out from gels and further cultured in 96 well low attachment plates (Sumilon, PrimeSurface 96U) as free floating vascular organoids for up to 3 months.

Reprogramming and characterization of human iPSCs. Human dermal fibroblasts (ATCC) and blood samples were reprogrammed as previously described (Agu et al. Stem cell reports 5, 660-71 (2015)). To check chromosomal integrity, multiplex-fluorescence in situ hybridization (M-FISH) was performed as described by Agu et al. For Genotyping, sample preparation was performed according to Infinium HTS Protocol Guide as recommended by Illumina Inc. The genotyping was performed using the Illumina Infinium PsychArray-24 BeadChip scanned with the Illumina iScan systems according to manufacturer's instruction. Genotypes were called using Illumina GenomeStudio (Illumina, San Diego, Calif., USA) with Genotyping software (Module 2.0.1), excluding samples with call rate <0.995. For analysis we applied the default settings by Illumina, the InfiniumPsychArray-24v1-1 Al manifest and Infinium PsychArray-24v1-1 Al ClusterFile cluster files. The CNV analysis and plotting were performed by using the bcftools cnv.

Immunocytochemistry. Vascular networks in Collagen I:Matrigel gels were fixed for 20min and free floating vascular organoids fixed for lh with 4% PFA at room temperature (RT) and blocked with 3% FBS, 1% BSA, 0.5% Triton, and 0.5% Tween for 2 h at RT on a shaker. Of note, vascular organoids are more stable than the initially formed vascular networks in 3D gels and therefore could be used for standard immunohistochemistry procedures. Primary antibodies were diluted 1:100-1:200 in blocking buffer and incubated over night at 4° C. The following antibodies were used in these study: anti-CD31 (DAKO, M082329), anti-VE-Cadherin (Santa Cruz, sc-9989), anti-ICAM-1 (Sigma, HPA002126), anti-PDGFR-β (CST, 31695), anti-SMA (Sigma, A2547), anti-Calponin (Abcam, AB46794), anti-Collagen Type IV (Merck AB769), anti-Laminin (Merck, 19012), anti-MYH11 (Sigma HPA014539). After 3×10 min washes in PBS-T (0.05% Tween) the samples were incubated with the corresponding secondary antibodies from Life Technologies: Alexa Fluor 555 donkey-anti-mouse (A31570), Alexa Fluor 647 donkey-anti-rabbit (A31573), Alexa Fluor 488 donkey-anti-goat (A11055), Alexa Fluor 488 donkey-anti-sheep (A11015) at 1:250 in blocking buffer for 2h at room temperature. After 3 20 min washes in TBST the samples were counterstained with DAPI. The samples were mounted (DAKO S302380), dried overnight and imaged subsequently with a Zeiss 780 Laser Scanning Microscope. Vascular organoid transplantation. Vascular organoids were transplanted under the kidney capsule of 12-15 weeks old NSG mice. All surgical procedures were done accordingly to the Austrian law and ethical approval. Mice were imaged using MRI to monitor the transplant over time. To test perfusion of the human blood vessel implants, mice were injected i.v. with either FITC-Dextran (1.25mg/mouse, Invitrogen D1822) or anti-human CD31-Alexa 647 (2pg/mouse, BD 558094). Excised transplants were fixed with 4% PFA for 2 h at RT and stained as a whole as described for the vascular organoids above or processed for immunohistochemistry or standard H&E histology. To distinguish between the endogenous mouse and transplanted human vasculature, a specific anti-human CD31 antibody (DAKO, M082329) was used and to visualize the murine blood vessels, we used a specific anti-mouse CD31 antibody (Abcam, AB56299). To exclude possible cross-reactivity, these antibodies were tested on both human and mouse control sections, validating specificity. Samples were imaged with a Zeiss 780 Laser Scanning Microscope.

MRI imaging. MRI was performed on a 15.2 T Bruker system (Bruker BioSpec, Ettlingen Germany) with a 35 mm quadrature birdcage coil. Before imaging, the tail line was inserted for delivery of contrast agent (30-gauge needle with silicon tubing). All animals (N=3) were anaesthetised with isoflurane (4% induction, maintenance with 1.5%). During imaging, respiration was monitored and isoflurane levels were adjusted if breathing was <50 or >80 breaths per minute. Mice were kept warm with water heated to 37° C. circulated using a water pump. For anatomical localization and visualization of the implant a multi-slice multi-echo (MSME) spin echo sequence was used (repetition time (TR)/echo time (TE)=3000/5.8−81.18 ms, 14 echoes, 117 μm2 in-plane resolution, 0.5 mm slice thickness, number of experiments [NEX]=1). A pre-bolus injection of 0.05 ml of 0.01 mol/l gadolinium-based contrast agent (Magnevist, Berlex) was injected in order to correct for the contrast agent leakage. Dynamic susceptibility contrast (DSC) perfusion MRI was collected using fast imaging with steady-state precession (FISP) with 500.6 ms temporal resolution (1 slice; TR/TE=500/1.7 ms; flip-angle=5 degrees; 468×468 μm² in-plane resolution; 1-mm slice thickness; NEX=2; 360 repetitions) following tail-vein injection of 0.05 mL of 0.25 mol/L Magnevist. DSC data were used to calculate perfusion, relative blood volume (rBV), mean transit time (MTT) and leakage (K2). Processing was done offline using ImageJ (National Institutes of Health; rsbweb.nih.gov/ij/), and the DSCoMAN plugin (Duke University, dblab.duhs.duke.edu/wysiwyg/downloads/DSCoMAN 1.0.pdf). The analysis consisted of truncating the first 5 time points in the DSC-MRI time series to ensure steady-state magnetization, calculating the pre-bolus signal intensity (S₀) on a pixel-wise basis, converting the truncated DSC-MRI time series to a relaxivity-time curve (ΔR₂*(t)=−(1/TE)ln(S(t)/S₀) is dynamic signal intensity curve, and correcting for the gadolinium leakage (K₂) as described previously (Boxerman et al. Am. J. Neuroradiol. 27, 859-867 (2006)).

Modeling diabetic vasculopathy in human vascular organoids. Established endothelial networks in vascular organoids were cultured in a non-diabetic control medium (17 mM Glucose) or diabetic medium (75 mM Glucose, in the presence of absence of human TNFα (1 ng/mL, Invitrogen PHC3011) and/or IL-6 (1 ng/mL, Peprotech 200-06)) for up to 3 weeks before the basement membrane was investigated by Collagen type IV immunostaining and electron microscopy. D-Mannitol was used in non-diabetic media to control for hyperosmotic effects. For basement membrane quantifications, acquired z-stacks were analysed and the thickness of ColIV coats around luminal structures were measured using ImageJ software. For drug treatment, organoids were exposed to diabetic medium (75 mM Glucose, 1 ng/mL human TNFα, and 1 ng/mL IL-6) in the presence or absence of the following drugs: 2,4-Thiazolidinedione (5 mM, Abcam ab144811), Metformin (5 mM, Abcam ab120847), Acarbose (80 μg/mL, Sigma A8980), Nateglindine (100 μM, Sigma N3538), Diphenyleneiodonium (10 μM), Glimepiride (30 nM, Sigma G2295), Pioglitazone (10 μM, Sigma E6910). The following small molecule inhibitors were used: N-Acetyl-L-cysteine (500 μM, Sigma, A7250), CHIR99021 (10 μM, Tocris 4423), Goe6976 (100nM, Merck U51365250), MK2206 (10 μM, EubioS1078), QNZ (10 μM, Eubio S4902), 5B203580 (10 μM, Eubio S1076), SCH772984 (500 nM, Eubio S7101), 5P600125 (10 μM, Eubio 51460) Y-27632 (10 μM, Calbiochem 688000), DAPT (25 μM, Sigma D5942), 5B431542 (10 μM, Abcam ab120163).

FACS analysis of vascular organoids. Non-diabetic and diabetic vascular organoids were disaggregated using 25 μg/mL Hyaluroniduase (Worthington), 3 U/mL Dispase (Gibco), 2 U/mL Liberase (Roche) and 100 U DNAse (Stemcell Tech) in PBS for 45-60 min at 37° C. Subsequently, single cells were stained with the following antibodies: anti-CD31 (BD, 558094), anti-PDGFR-β (BD, 558821), anti-CD90 (Biolegend, 328117), anti-CD45 (ebioscience, 11-0459-41), and anti-CD73 (BD 742633). DAPI staining was used to exclude dead cells. A BD FACS Aria III was used for cell sorting and a BD FACS LSR Fortessa II for cell analysis.

Genome editing using CRISPR/Cas9. A mammalian expression vector expressing Cas9 from S. pyogenes with a 2A-Puro cassette¹³ (Addgene Plasmid: #62988;) was cut with BbSI (Thermo Fisher ER1011) shortly after the U6 promoter. Subsequently the plasmid was re-ligated introducing sgRNAs for either Neurogenic locus notch homolog protein 3 (Notch3) or Delta-like protein 4 (D114). The following primers were used for sgRNA annealing: Notch3: forward, caccgGCCACTATGTGAGAACCCCG (SEQ ID NO: 7); reverse, aaacCGGGGTTCTCACATAGTGGCc (SEQ ID NO: 8); D114: forward, caccg-CAGGAGTTCATCAACGAGCG (SEQ ID NO: 9); reverse, aaacCGCTCGTT-GATGAACTCCTGc (SEQ ID NO: 10). sgRNA plasmids were verified by

Sanger sequencing and used for electroporation of iPSCs (NC8) with the 4D-Nucleofector System (Lonza). 2 μg plasmid DNA were transfected using the P3 Primary Cell 4D-Nucleofector Kit. Transfected NC8 cells were seeded on Matrigel coated 6-well plates in Essential 8 Media (Gibco) containing 50 μM Y27632 (Calbiochem) and cultured for 24 hours before Puromycin treatment (0.2 μg/ml) for 48 hours. Remaining cells were cultivated until colony formation could be observed and single colonies were further expanded for genotyping with Sanger sequencing. Knock-out cell lines were verified by Western Blot or immunofluorescence staining.

Modeling diabetic vasculopathy in human vascular organoids in vivo. Immunodeficient NSG mice carrying human vascular organoid transplants were daily i.p. injected with 40 mg/kg Streptozotocin (STZ) (Merck, 572201) for 5 consecutive days. Every day, STZ was freshly dissolved in citrate buffer (pH4.6) and immediately used. Diabetes was confirmed (blood glucose >300 mg/dL) by measuring non-fasting glucose using the OneTouch UltraEasy system (Lifetouch, AW 06637502C). DAPT (Selleckchem 52215) was dissolved in ethanol and injected with 90% cornoil for 5 consecutive days at 5 mg/kg with 2 days of no treatment per week. Anti-Notch3 blocking antibodies (R&D AF1559) were injected 3 time/week at lmg/kg. For quantifying vessel leakage, the FITC-Dextran⁺ area was measured and normalized to the area of perfused human blood vessels (hCD31⁺) using the FIJI software. This ratio was then further normalized to control non-diabetic mice. Permeability of long-term DAPT treated vessels were measured after 2 days of treatment stop to avoid measuring acute effects of DAPT on vessel permeability.

Next Generation Sequencing and qRT-PCR analysis. Vascular networks of non-diabetic and diabetic vascular organoids were disaggregated using 25 μg/mL Hyaluroniduase (Worthington), 3 U/mL Dispase (Gibco), 2 U/mL Liberase (Roche) and 100 U DNAse (Stemcell Tech) in PBS for 45-60 min at 37° C. Subsequently, single cells were stained for CD31 expression (BD 558094) and DAPI negative (=alive cells) were FACS sorted using an FACS Aria III machine. The CD31 positive, DAPI negative endothelial cells were directly sorted into Trizol LS buffer (Invitrogen) and processed further to RNA isolation. For RNA Seq mRNA was enriched by poly-A enrichment (NEB) and sequenced on a Illumnia HiSeq2500. For qRT-PCR analysis, total RNA was extracted from whole vascular organoids using Trizol (Invitrogen) and cDNA was synthesized using the iscript cDNA synthesis kit (Biorad), performed with a SYBR Green master mix (Thermo) on a Biorad CFX real time PCR machine. All data were first normalized to GAPDH and then compared to the non-diabetic control samples. The following primers were used:

Col4a1-FWD:
(SEQ ID NO: 1)
TGCTGTTGAAAGGTGAAAGAG
Col4a1-REV:
(SEQ ID NO: 2)
CTTGGTGGCGAAGTCTCC
Co14a2-FWD:
(SEQ ID NO: 3)
ACAGCAAGGCAACAGAGG
Co14a2-REV:
(SEQ ID NO: 4)
GAGTAGGCAGGTAGTCCAG
GAPDH-FWD:
(SEQ ID NO: 5)
TCTTCTTTTGCGTCGCCAG
GAPDH-REV:
(SEQ ID NO: 6)
AGCCCCAGCCTTCTCCA
FN1-FWD:
(SEQ ID NO: 11)
ACACAAGGAAATAAGCAAATG
FN1-REV:
(SEQ ID NO: 12)
TGGTCGGCATCATAGTTC
TUBB-FWD:
(SEQ ID NO: 13)
CCAGATCGGTGCCAAGTTCT
TUBB-REV:
(SEQ ID NO: 14)
GTTACCTGCCCCAGACTGAC

Bioinformatic analysis. RNA-seq reads were aligned to the human genome (GRCh38/hg38) using Tophat v2.0.10 and bowtie2/2.1.0, gene- and transcript-level abundance estimation in TPM, FPKM and expected counts was performed with RSEM v1.2.25, aligned reads were counted with HTSeq v0.6.1p1 and differential expression analysis was carried out using DESeq2 v1.10.1, with FDR threshold of 0.05. Enrichr (Kuleshov et al. Nucleic Acids Res. 44, W90-7 (2016)) was used to identify GO terms of up-regulated genes. The expression profile similarity search server-CellMontage v2 (cellmontage2.cira.kyoto-u.ac.jp; Fujibuchi et al. Bioinformatics 23, 3103-3104 (2007)), was used to classify the expression profiles of iPS.EC cells with respect to normal cell types. The average iPS.EC cells transcripts relative abundance in TPM was compared to the 2919 pre-processed human gene expression datasets. Based on the obtained results iPS.EC are most similar to endothelial cells—the 75 datasets with highest correlation are all derived from endothelial cells—55 stem from vein endothelial cells, 13 from microvascular endothelial cell, 5 from artery endothelial cell, 2 from lymphatic vessel endothelial cell, with correlation coefficients ranging from 0.69 to 0.64 and p-values ranging from 5.38e-2212 to 6.39e-1828. To compare iPS.EC expression profiles with published human tissue RNA-seq data (Lonsdale et al. Nat. Genet. 45, 580-5 (2013)), we combined our and available expression profiles as previously described (Danielsson et al. Brief. Bioinform. 16, 941-949 (2015).), using log-transformed F/RPKM values for 2338 tissue specific genes (Cavalli et al. Genome Biol. 12, R101 (2011)), removing sequencing study batch effects using ComBat, and inspecting sample clustering in a correlation heat map. Expression profiles of iPS.EC are found to cluster with those of GTEx arterial samples.

Skin samples from type II diabetes and normo-glycemic control patients. Surgical samples of human skin were taken from T2D and non-diabetic patients. Non-necrotic, healthy skin was taken from leg amputates. Leg amputations of T2D patients were necessary because of diabetic foot syndrome. Leg amputations of non-diabetic patients were performed as a consequence of accidents, venous ulcerations or other vascular diseases not related to T2D. The present study was approved by the local ethics committee and all included patients gave their informed consent (no. 449/2001; 81/2008). Of note, we included dermis that was isolated at the maximal possible distance of any ulcers or necroses of the leg amputates. The details of patient collectives are shown in the following Table 1.

TABLE 1
Comparison of patient characteristics and laboratory parameters
between patients with and without type 2 diabetes (T2D).

| Parameter | T2D | Control | P |
|---|---|---|---|
| n | 13 | 13 | — |
| Sex m/f | 5/8 | 4/9 | |
| Age (years, mean ± SD) | 54.6 ± 16 | 47.4 ± 15.5 | n.s.** |
| BMI (kg/m², | 27.9 ± 3.3 | 27.75 ± 3.4 | n.s.** |
| mean ± SD) | | | |
| Duration of T2D (years, | 9.1 ± 6.1 | — | |
| mean ± SD) | | | |
| HbA1c (%, mean ± SD)* | 8.0 ± 2.1 | 5.48 ± 0.34 | <0.001** |
| Creatinine (mg/dl, | 1.15 ± 0.6 | 0.80 ± 0.23 | n.s.** |
| mean ± SD) | | | |
| Smokers (n %) | 7 (54%) | 6 (46%) | |

Values are means ± SD.
n: number,
m: male,
f: female,
SD: standard deviation,
BMI: body mass index,
P: p-value,
n.s.: not significant,
**t-test,
*glycated haemoglobin in percent (%).

Immunohistochemistry in patient skin. Human skin material was either cryofixed in Geltol and stored at −80° C., or embedded in paraffin after 4% paraformaldehyde fixation. 2-5 μm sections were cut and used for subsequent immunofluorescence or immunohistochemical stainings. Paraffin sections were dewaxed, hydrated, and heat induced antigen retrieval was performed. Antigenicity was retrieved by microwaving (3×5 minutes, 620 W) or by heating the sections in an autoclave (60 minutes) in 10 mM citrate buffer (pH 6.0). Cryosections were stored at −20° C., thawed and dried at time of use and fixed in ice-cold acetone for 20 minutes. This was followed by incubation with indicated primary antibodies and visualized by a biotin-streptavidin-horseradish peroxidase dase method, or by using fluorescently labelled secondary antibodies.

Human patient-derived endothelial cell preparations. Four T2D and six non-diabetic patients were analyzed. For ex vivo preparations of BECs (spell out), a mechanical and enzymatic micropreparation protocol was used, including the use of Dispase I (Roche Inc., #210455) as previously described¹¹. The resulting single cell suspensions were blocked with 1×PBS-1% FCS and incubated with anti-CD31, anti-CD45 and anti-podoplanin antibodies in a three-step procedure with intervening washing steps. For antibodies see section above. Subsequently, cells were subjected to cell sorting using a FACStar Plus (Becton Dickinson). Total CD31⁺ podoplanin endothelial cells were separated, re-analyzed, twice pelleted (200 g), lysed in RLT buffer (Qiagen; #74104) and further processed for RNAseq.

Electron Microscopy. Vessel organoids were fixed using 2.5% glutaraldehyde in 0.1M sodium phosphate buffer, pH 7.2. for 1 h at room temperature. For electron microscopy of dermal blood vessel from diabetic and non-diabetic leg amputates, human skin was fixed in 4% PFA and 0.1% glutaraldehyde and embedded in Lowicryl-K4M. Samples were then rinsed with the same buffer, post-fixed in 1% osmium tetroxide in ddH2O, dehydrated in a graded series of acetone and embedded in Agar 100 resin. For electron microscopy of dermal blood vessel from diabetic and non-diabetic leg amputates, human skin was fixed in 4% PFA and 0.1% glutaraldehyde and embedded in Lowicryl-K4M. 70-nm sections were cut and post-stained with 2% uranyl acetate and Reynolds lead citrate. Sections were examined with an FEI Morgagni 268D (FEI, Eindhoven, The Netherlands) operated at 80 kV. Images were acquired using an 11 megapixel Morada CCD camera (Olympus-SIS).

Rodent models of diabetes. Rodent models used in this study and according references are listed in Table 2. Controls were either age matched WT animals or untreated strains as indicated in the Table. Sections of paraffin embedded skin samples of all rodent models were stained HE and PAS to visualize vessel morphology.

TABLE 2
Overview of rodent models analysed in our study.

| Species | Strain | Genetics/model | Control | Type | Duration | | Phenotype |
|---|---|---|---|---|---|---|---|
| Mouse | C57 | lepr db/db | WT | genetic | 28 | weeks | type 2 |
| | BL/KsJ | | | | | | diabetes |
| Mouse | C57 | lep ob/ob | WT | genetic | 28 | weeks | type 2 |
| | BL/KsJ | | | | | | diabetes |
| Mouse | C57 | STZ | WT | chemical | 17 | weeks | type 1 |
| | BL/KsJ | | | | | | diabetes |
| Mouse | C57 | STZ | WT | chemical | 24 | weeks | type 1 |
| | BL/KsJ | | | | | | diabetes |
| Mouse | C57 | Insr ko + | no | transgenic | 24 | weeks | type 2 |
| | BL/KsJ | doxycycline | doxycycline | | | | diabetes |
| Mouse | C57 | LDLR ko + | normal diet | transgenic | 16 | weeks | obese |
| | BL/6 | high fat + glu- | | | | | |
| | | cose diet | | | | | |
| Mouse | C57 | high fat diet | WT | diet | 16 | weeks | type 2 |
| | BL/6 | | | | | | diabetes |
| Rat | ZDF | Lepr, zucker dia- | lean littermates | genetic | 6 | months | type 2 |
| | | betic fatty | | | | | diabetes |
| Rat | SD | STZ | WT | chemical | 6 | months | type 1 |
| | | | | | | | diabetes |

(References: Goldman, O. et al. Stem Cells 27, 1750-1759 (2009); Watabe, T. et al. J. Cell Biol. 163, 1303-11 (2003); Potente et al. Cell 146, 873-887 (2011); Kern et al. Am. J. Physiol. Metab. 280, E745-51 (2001); Pickup et al. Life Sci. 67, 291-300 (2000); Wellen et al. Journal of Clinical Investigation 115, 1111-1119 (2005); Li et al. J. Diabetes Complications 29, 568-571 (2015); Lieb, W. et al. Circ. Cardiovasc. Genet. 3, 300-306 (2010); Lim et al. Atherosclerosis 180, 113-118 (2005))

Statistics. If not otherwise stated, all values are presented as means±SEM. Statistical analyses were performed using GraphPad Prism. All statistical tests used are described in the figure legends. P<0.05 was accepted as statistically significant.

### Example 2

### Establishment of Human 3D Blood Vessel Organoids

Capillaries are composed of endothelial cells that form the inner lining of the wall and pericytes that are embedded within a surrounding basement membrane. Human endothelial cells have been previously derived from human embryonic stem cells (hESCs) and from induced pluripotent stem cells (iPSC) (James et al. Nat Biotechnol 28, 161-6 (2010); Patsch et al. Nat. Cell Biol. 17, 994-1003 (2015)). Moreover, perivascular cells such as vascular smooth muscle cells can be generated from human ESCc and iPSCs (Cheung et al. Nat. Biotechnol. 30, 165-73 (2012)). However, to investigate mechanisms of complex vascular diseases, there is a need for highly sophisticated models that resembles all features of a human microvasculature such as a lumenized endothelium, endothelial-pericyte interactions and the formation of a common basement membrane and is applicable to high throughput drug screening. We therefore set out to establish 3D-human blood vessel organoids from hESC and iPS cells.

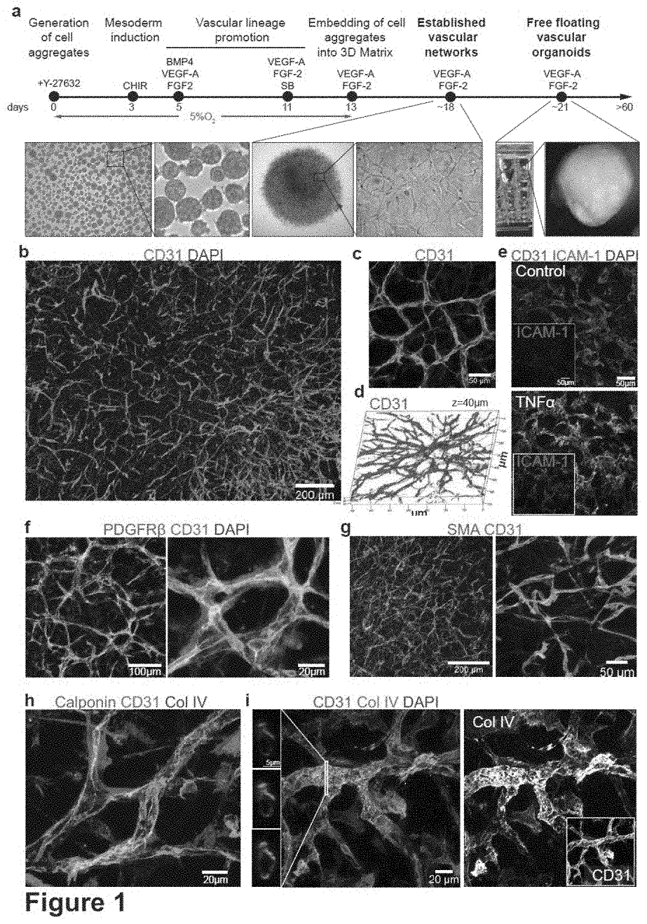

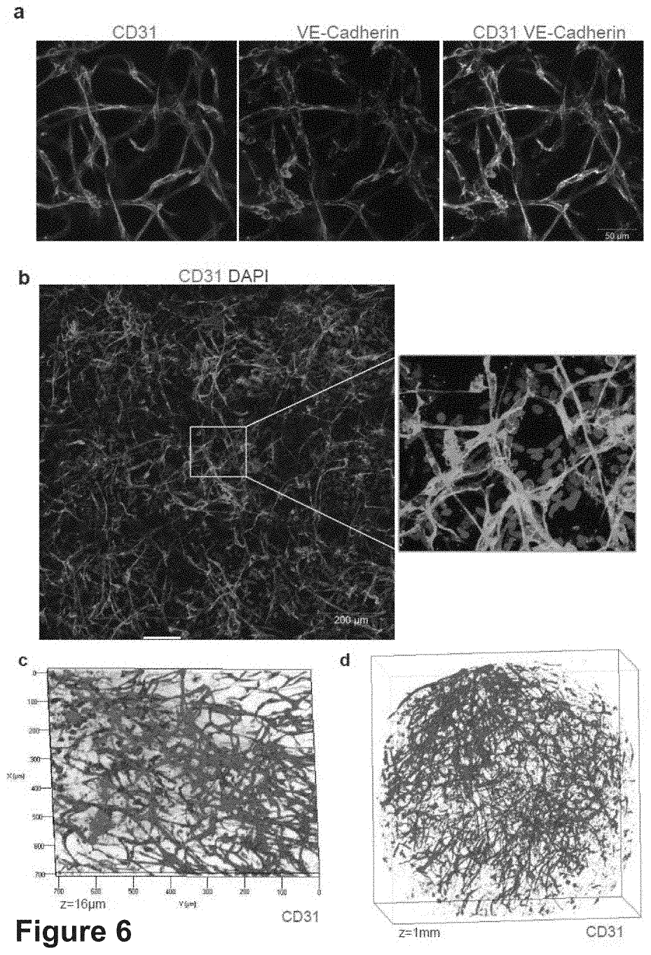

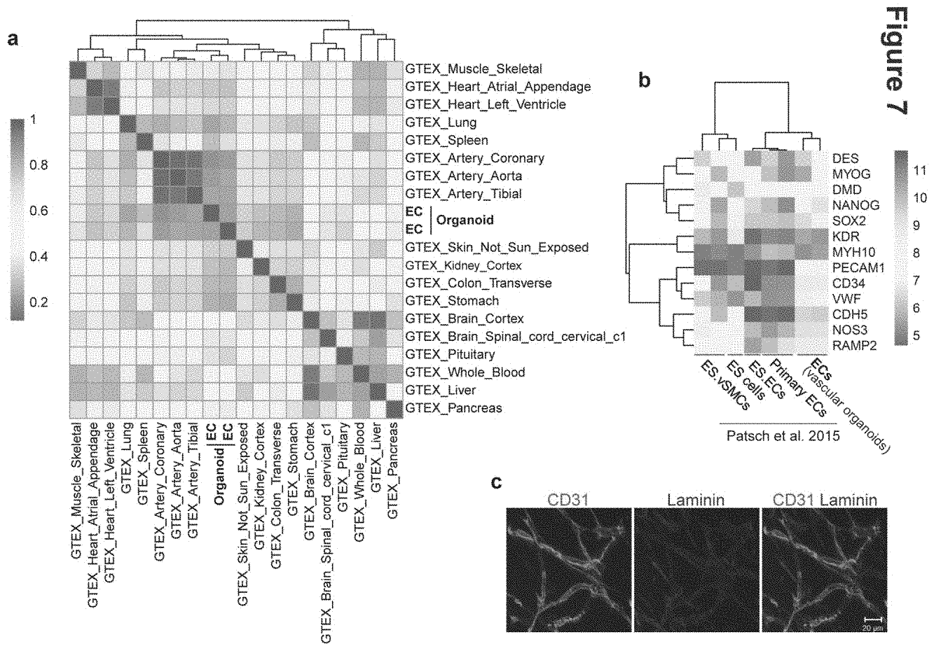

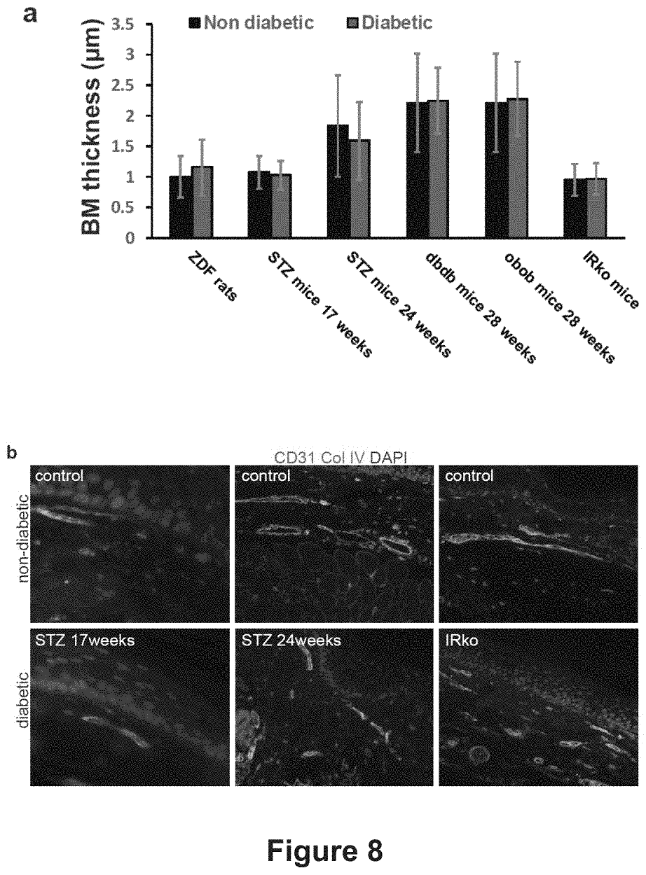

To accomplish this, we developed a multistep protocol to modulate signaling pathways involved in mesoderm development and blood vessel specification (FIG. 1). We first cultured human ES cell aggregates under low oxygen conditions and induced mesoderm differentiation by exposing these cell aggregates to the GSK inhibitor CHIR99021 to activate the Wnt pathway (Pasch et al., supra; Sumi et al. Development 135, 2969-2979 (2008)). Subsequently, cell aggregates were treated with BMP4, VEGF-A and FGF-2 to promote vascular differentiation (Bai et al. J. Cell. Biochem. 109, 363-374 (2010); Goldman et al. Stem Cells 27, 1750-1759 (2009)), followed by VEGF-A, FGF-2 and 5B431542 (to block TGFβ, signaling) (James et al., supra; Watabe et al. J. Cell Biol. 163, 1303-11 (2003)). These cell aggregates were then embedded in a 3D matrix and further stimulated with VEGF-A and FGF-2 to drive blood vessel differentiation. Following multiple pilots to test defined experimental conditions, we developed a 3D Matrigel/Collagen I matrix that allowed for the highly reproducible outgrowth of structures resembling vascular trees (FIG. 1a). Immunostaining for the vascular marker CD31 confirmed that these outgrowths contained a high proportion of endothelial tubes (FIG. 1b, c). We also observed staining for VE-Cadherin (FIG. 6a), an additional prototypical marker for endothelial cells. Confocal imaging showed the formation of a complex, interconnected network of CD31⁺ endothelial structures (FIG. 1d). We were also able to develop CD31⁺ blood vessel organoids from human iPSCs using this approach (FIG. 6b-d).

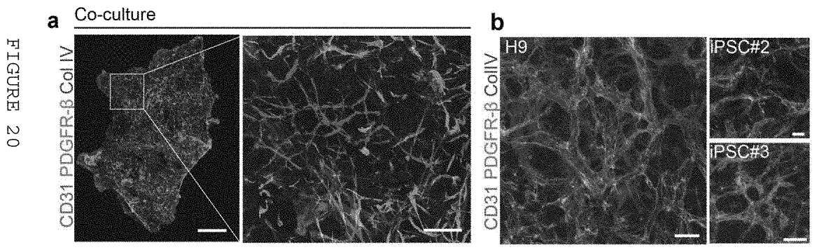

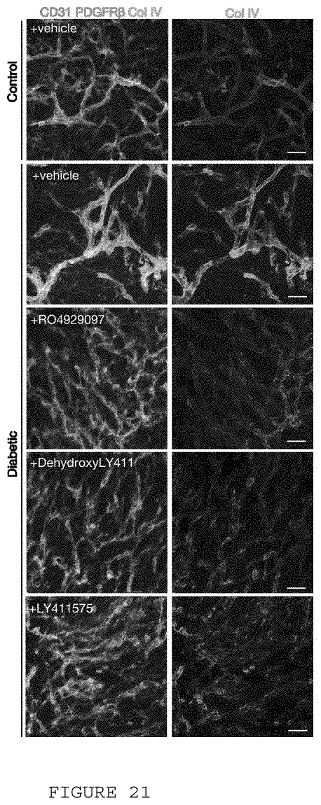

Next, we verified that the CD31⁺ blood vessel organoids recapitulated features of human blood vessels in vivo. To profile gene expression, we disaggregated the organoids, sorted for CD31⁺ endothelial cells and performed RNAseq. The gene expression profile of CD31⁺ endothelial cells from our 3D cultures indeed clustered most closely with gene expression profiles (GTEx) of human blood vessels (FIG. 7a). SHOGoin Cellmontage2 analysis further showed that our organoid endothelial cells exclusively matched human endothelial cells (not shown). Moreover, the endothelial cells isolated from organoids did not express the prototypic hESC markers SOX2 and Nanog, nor the smooth muscle markers dystrophin, desmin, and myogenin; importantly, however, they did express biomarkers of primary human endothelial cells or of previously reported 2D in vitro human endothelial cultures, such as CD34, CDHS, vWF, PECAM1, NOS3, or RAMP2 (FIG. 7b). The endothelial cells isolated from organoids also responded to TNFα stimulation by inducing the cell adhesion molecule ICAM1 (FIG. 1e), indicating their functional competence. Most importantly, these 3D vascular organoids were self-organizing, and we observed the formation and proper localization of pericytes as defined by the molecular markers CNN1, SMA, and PDGFRβ (FIG. 1f-h). The 3D structures were also surrounded by a basal lamina as determined by immunostaining for the prototypic vascular basal membrane markers Collagen IV (FIG. 1h,i) and Laminin (FIG. 7c). Of note, co-cultering of purified, differentiated endothelial cells and pericytes under the same conditions resulted in tenuous endothelial networks that showed only few pericyte interactions and were not covered by Collagen IV (FIG. 20a). Importantly, we were able to reproducibly generate similar 3D vascular networks using the human embryonic stem cell line H9 as well as two additional iPSC lines tested (FIG. 20b).

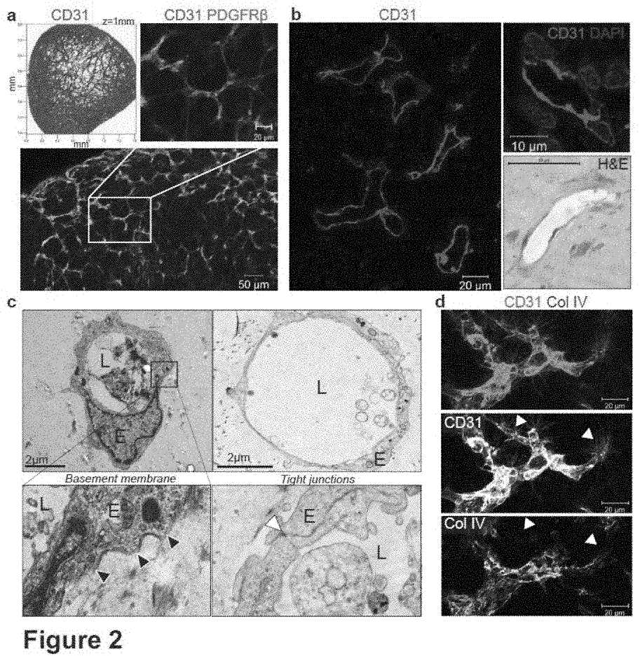

To further improve and standardize these in vitro microvasculatures for drug screening approaches and, we developed free-floating 3D organoid cultures in a 96 microwell format (FIG. 1a). These 1-2 mm free-floating organoids formed complex, branched 3D capillary networks consisting of CD31⁺ endothelial cells together with tightly associated pericytes (FIG. 2a). The generation of free-floating organoids from human ESCs and from iPSCs was robust and reproducible. Importantly, free-floating organoid cultures enabled the isolation of single organoids into wells for processing by immunohistology and electron microscopy (EM). Immunohistology and EM imaging indeed showed the formation of stereotypical capillaries with endothelial cells, pericytes and basement membrane, as well as the formation of typical tight junctions between endothelial cells (FIG. 2b, c). We also observed CD31⁺ tip cells at the edge of the growing blood vessels, indicative of a newly forming blood vessel (FIG. 2d). As expected, these tip cells are not surrounded by the basal membrane which only forms in mature capillaries (FIG. 2d). Free-floating vascular organoids expanded in culture for about 3-4 weeks, followed by growth arrest, and could be maintained for at least two months thereafter.

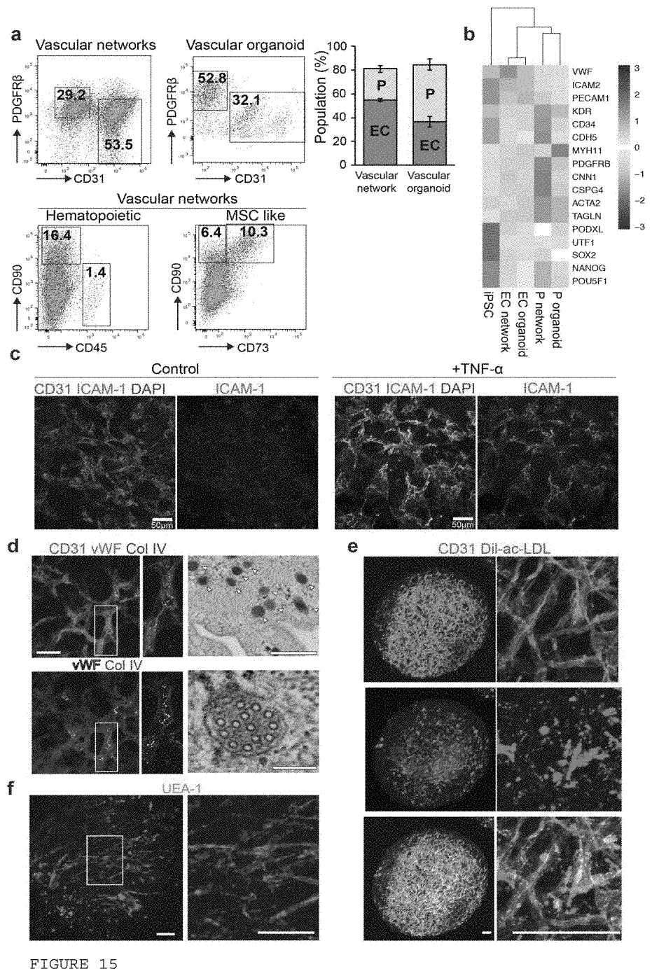

Next, we assessed the cellular composition of the vascular networks and free-floating organoids using FACS. Both contained PDGFR-β⁺ pericytes as well as CD31⁺ endothelium, to varying extents. The remaining cells were primarily CD90⁺ CD73⁺ mesenchymal stem like cells and a minor population of CD90⁻CD45⁺ haematopoitic cells (FIG. 15a). The gene expression profiles of CD31⁺ endothelial cells isolated from from our vascular networks and free-floating organoids confirmed that these cells express mature endothelial markers such as von-Willebrand factor (vWF) and efficiently downregulated the parental iPSC pluripotency markers (FIG. 15b). PDGFR-β⁺ isolated cells from the 3D cultures displayed a mixed endothelial/pericyte marker expression at the early vascular network stage which changed in the vascular organoids towards a typical pericyte signature such as expressing of NG2 (GSPG4), SMA(Acta2) or Calponin-1 (CNN1) (FIG. 15b). At this stage we also found some expression of Oct-4 and Nanog which might arise from early PDGFR-β⁺ pericyte progenitors that could be still present in the vascular organoid. Importantly, endothelial cells in our free floating organoids responded to TNF-α stimulation by inducing the cell adhesion molecule ICAM1 (FIG. 15c), reflecting functional competence. Moreover, we observed immunostaining for von Willebrand factor (vWF) and the generation of Weibal-Pallade bodies, uptake of acetylated LDL, as well as staining with the lectin UEA-1 (FIG. 15d-f), all indicative of mature endothelial cells. Thus, we have established self-organizing 3D human blood vessel organoids from hESCs and iPSCs that exhibit the morphological features and molecular signatures of bona fide human microvasculature.

Thus, we have established self-organizing 3D human blood vessel organoids from hESCs and iPSCs that exhibit the morphological features and molecular signatures of bona fide human microvasculature.

### Example 3

### Blood Vessel Organoids Establish Functional Human Vasculature in Mice

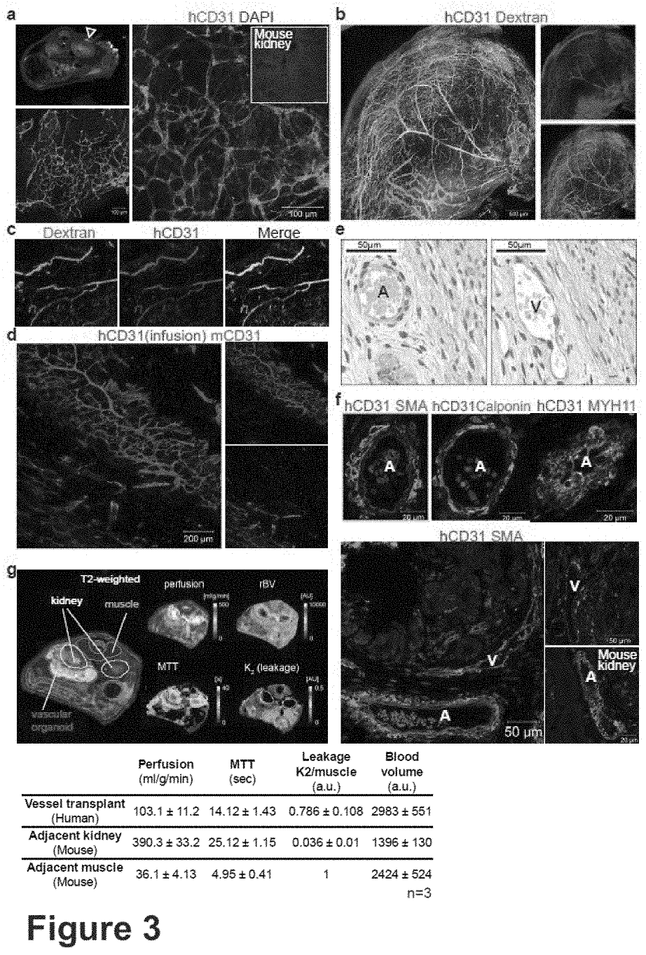

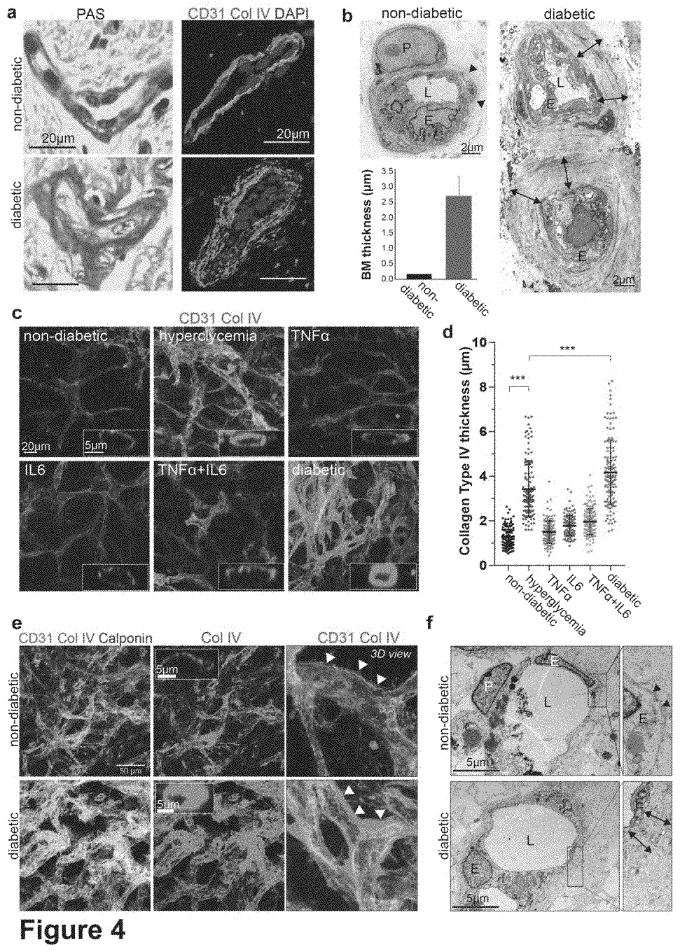

To test whether the blood vessel organoids can form functional blood vessels in vivo, we differentiated hiPSCs and hESCs into blood vessel organoids in vitro and transplanted them under the kidney capsule of immunodeficient host mice. The human organoids, because of their compact structure, could reproducibly be transplanted and indeed grew and survived in the mouse environment, in some cases for more than 6 months. We stained organoid and kidney tissue with human-specific anti-CD31, which showed that human vasculature had been established beside endogenous mouse blood vessels (FIG. 3a). We also observed sprouting of the human blood vessel, as determined by the formation of tip cells and growth of the human blood vessels into the adjacent tissue. To evaluate blood circulation, we perfused the recipient mice with FITC-Dextran. We found that the human blood vessels had gained access to the endogenous mouse vasculature (FIG. 3b,c). Similarly, when we perfused mice with human-specific anti-CD31 antibody, we observed perfusion and staining of blood vessels that were distinct from endogenous mouse vasculature, as determined by murine-specific anti-CD31 staining (FIG. 3d).

Importantly, histological sectioning showed that these human blood vessels had specified into arterioles, capillaries and venules (FIG. 3e). This specification was further confirmed using immunohistochemistry for human CD31⁺ endothelium, and for smooth muscle actin (SMA) or Calponin as markers of smooth muscle cells surrounding the endothelium (FIG. 3f). We also detected smooth muscle cells with an antibody specific for human myosin heavy chain 11 (MYH11) (FIG. 3f). Moreover, perfusion of the transplanted human blood vessel organoids was confirmed with MRI imaging, which detected flow into the transplanted human blood vessel organoids, and importantly, also exit of the blood from the vascular tree (FIG. 3g). Quantitative MRI measurements for perfusion rates and blood volumes showed well vascularized and perfused transplants; moreover, the mean transit times (MTT) and low vessel leakage (K₂) confirmed apparently normal organization and function of the human blood vessels, as compared to blood flow parameters in the adjacent endogenous mouse kidney and muscle (FIG. 3g). These data show that our human 3D capillary organoids can specify into arterioles and venules in vivo, and form a perfused, functional human vasculature in recipient mice.

### Example 4

### Diabetic Vasculopathy in Human Blood Vessel Organoids

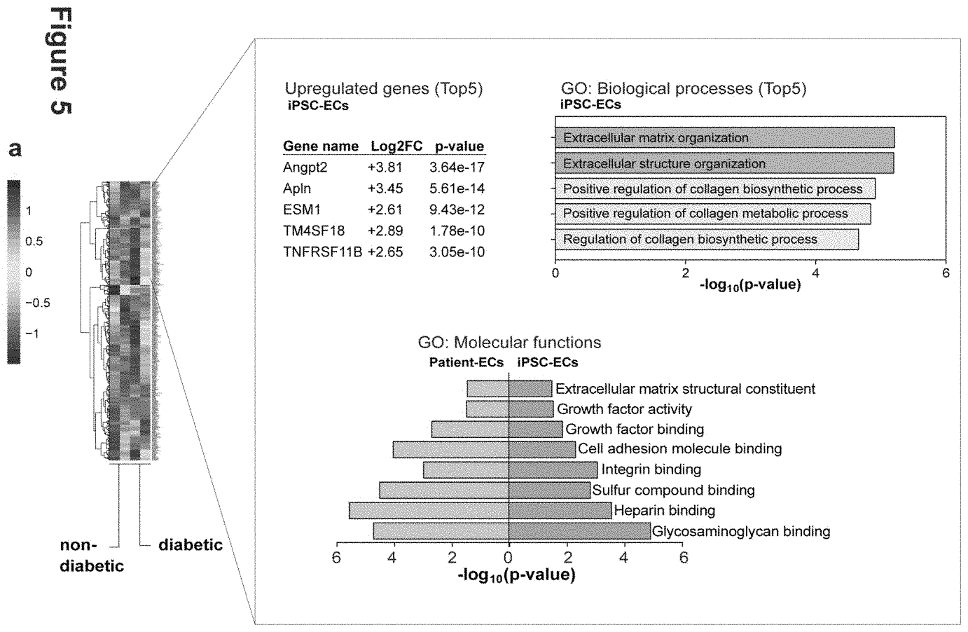

Diabetes is a major cause of blindness, kidney failure, heart attacks, stroke or lower limb amputation; in large parts because of marked changes in blood vessels, defined by expansion of the basal membrane. Such structural changes due to diabetic microangiopathy have been observed in the human kidney or muscle biopsies. To confirm diabetic microvascular changes in humans, we examined the dermal skin microvasculature in surgical specimens of normo-glycemic individuals and type 2 diabetic (T2D) patients. The clinical characteristics including age, sex, body-mass index (BMI), serum creatinine levels, and years of disease are shown in Table 1. In dermal blood vessels of normo-glycemic controls, the CD31⁺ capillary endothelium was surrounded by a thin basal membrane, as determined by collagen IV and by PAS staining (FIG. 4a) and electron microscopy (FIG. 4b). The dermal microvasculature of all type 2 diabetic patients, included in our study, revealed prominent alterations in the deposition of extracellular cellular matrix proteins and massively thickened, onion-skin-like lamination and typical splitting of the basement membrane layer (FIG. 4a,b). Thus, we observe massive basement membrane thickening of dermal capillaries of T2D patients, as expected.

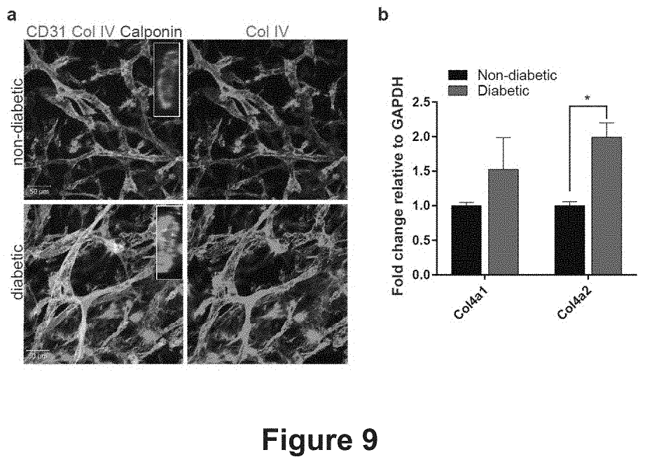

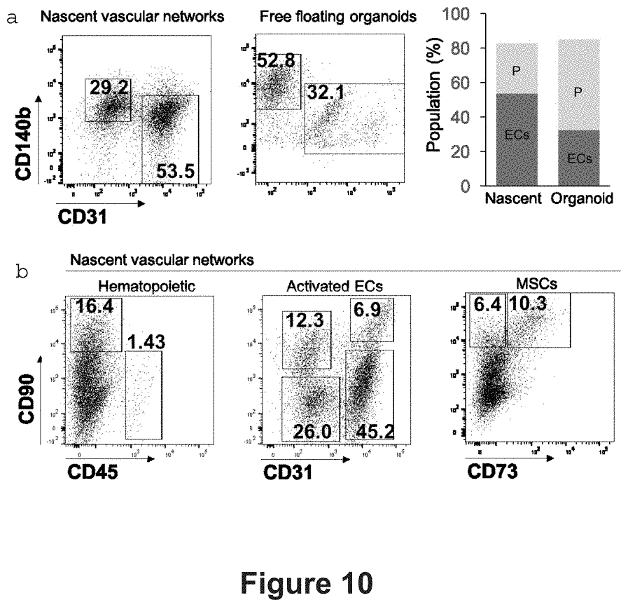

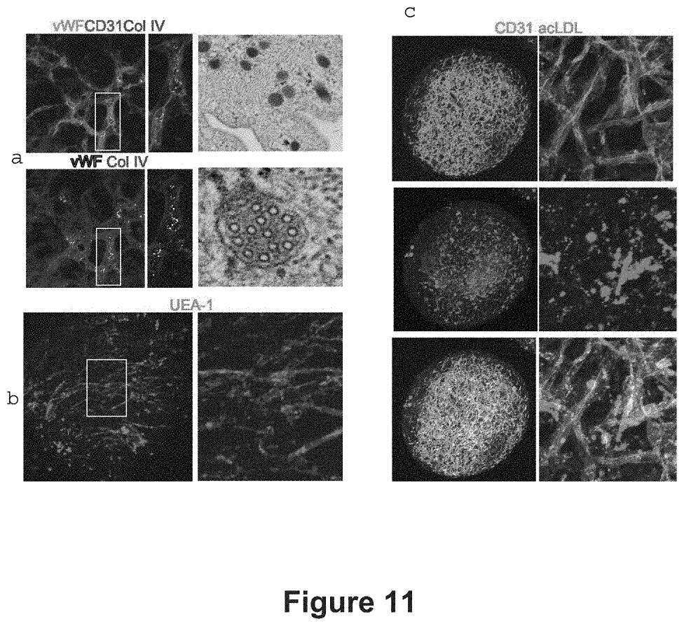

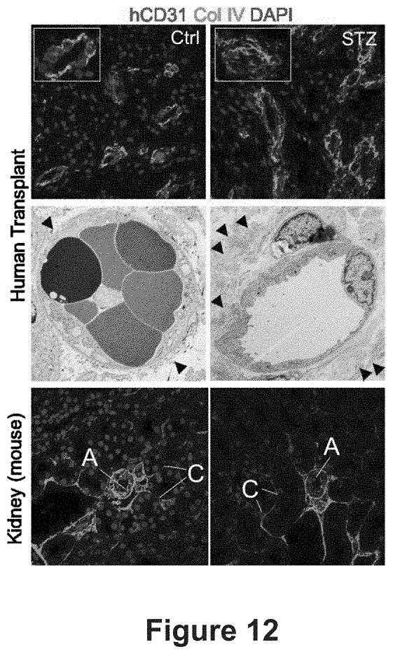

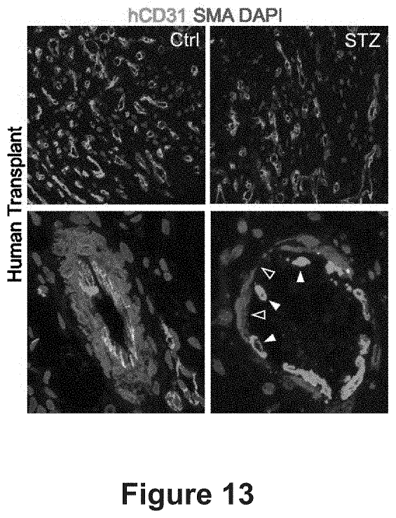

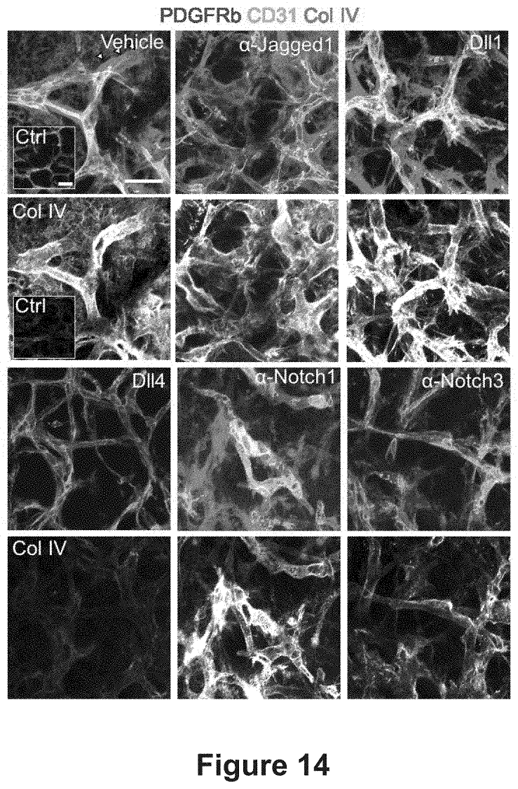

Although diabetic microangiopathy is evident in the skin of diabetic patients, it has not been observed previously in various rodent models of diabetes. We thus performed a comprehensive evaluation of the skin microvasculature of multiple genetic and environment-induced mouse and rat models of diabetes. However we failed to detect increased basement membranes indicative of dermal vasculopathy in any of these models, including leptin and leptin receptor mutant ob/ob and db/db mice, streptozotocin-treated mice, doxycycline induced knock-out of the insulin receptor in mice, high fat diet, high fat and glucose in LDLR mutant mice, or Zucker diabetic fatty rats, carrying a leptin receptor mutation, and streptozotocin treated rats (FIG. 9a,b, and Table 2). Thus, none of these very severe and long-term rodent models of type 1 and type 2 diabetes exhibited a key hallmark of human diabetic vasculopathy, thickening of the blood capillary basal membrane.

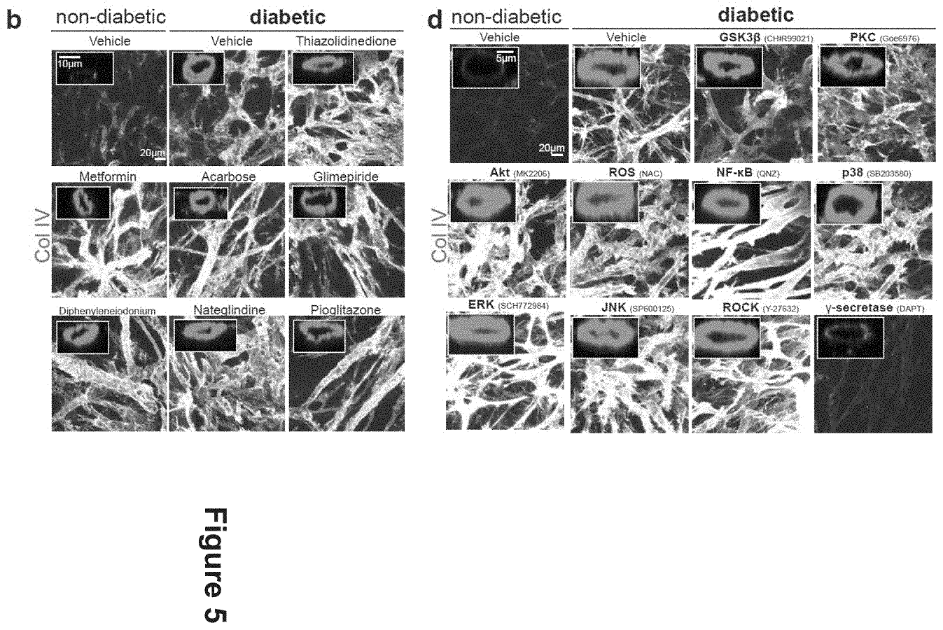

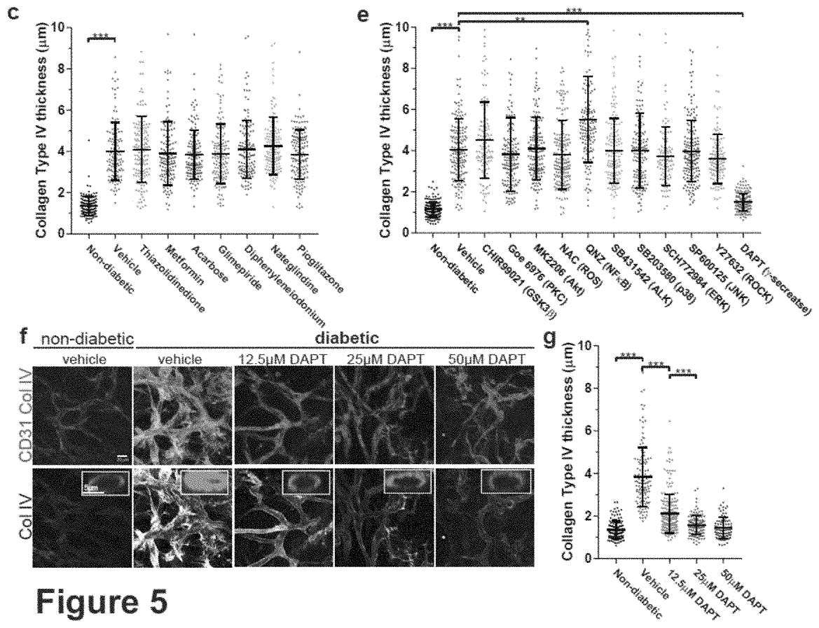

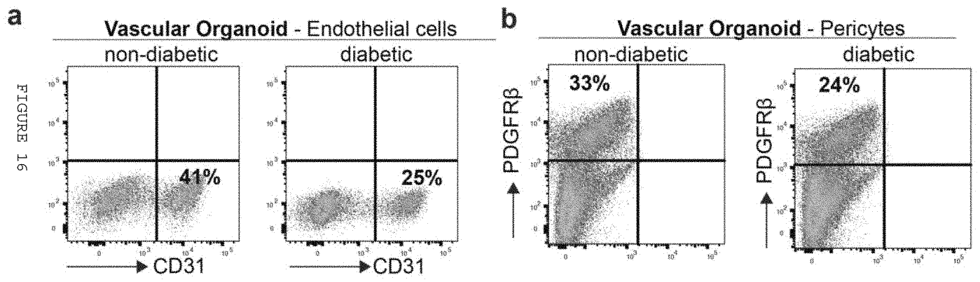

Given that rodent models of diabetes do not recapitulate the dermal vasculopathy present in diabetic patients, we wanted to test if we could model this crucial phenotype in our human blood vessel organoids. To accomplish this, we cultured our 3D blood vessel organoids in medium with elevated glucose and monitored expansion of the basement membrane as detected by collagen IV, similarly to the human skin samples. Intriguingly, hyperglycemia resulted in a significant increase in collagen IV in human blood vessel organoids (FIG. 4c,d). Since diabetes is accompanied by an inflammatory state, including elevated serum levels of proinflammatory cytokines such as TNF-α and IL-6, we also cultured blood vessel organoids with or without TNFα and IL6 under normoglycemic or hyperglycemic conditions. Under normo-glycemic conditions, collagen IV expression in blood vessel organoids was not significantly altered by exposure to TNFα and IL6, either alone or in combination. However, collagen IV deposition was markedly enhanced in blood vessel organoids exposed to elevated glucose and both TNF-α and IL-6 (FIG. 4c,d). Confocal cross-sectioning of these organoids confirmed the marked expansion of collagen IV and thickening of basal membrane in response to treatment with a “diabetic cocktail” of TNFα, IL6 and high glucose (FIG. 4e). Moreover, consistent with our findings with human T2D patients (FIG. 4b), we observed a massive thickening and splitting of the basement membrane layer by EM (FIG. 4f). This thickening of the vascular basement membrane in response to the diabetic cocktail was observed with blood vessel organoids derived from human iPSCs (FIG. 4c-f) as well as from human ESCs. Next, we characterized the “diabetic organoids” exposed to high glucose/TNFα/IL6 based on gene expression. We observed a marked reduction of endothelial cells as well as pericyte loss in the vascular organoids exposed to TNF-alpha, IL6 and high glucose (FIG. 16a,b). We performed RNAseq on CD31⁺ endothelial cells sorted from control and diabetic human blood vessel organoids. Genes previously implicated as markers for diabetes in humans, including Angiopoietin 2, Apelin, ESM1, and TNFRSF11B, were among the top five most upregulated genes in diabetic organoids relative to control organoids (FIG. 5a). Indeed, the differential gene expression profiles generated from diabetic vs. control organoids, as well as from T2D patient vs. dermal blood vessels from normo-glycemic individuals, revealed a marked overlap among the diabetic organoids and T2D patients, annotated to extracellular matrix, cell adhesion, and growth factor activity/binding (FIG. 5a). We also found increased mRNA levels of collagen IV in the diabetic organoids compared to controls (FIG. 9b), and the top five gene ontology (GO) pathway terms of differentially expressed genes between CD31⁺ endothelium from control and diabetic organoids were all associated with collagen biosynthesis and extracellular matrix reorganization (FIG. 5a). In contrast to blood vessel organoids, various endothelial cells exposed to high glucose with or without TNFα/IL6 did not upregulate extracellular matrix and collagen biosynthesis components, including HUVECs, the immortalized human microvascular endothelial cell line HMEC1, and primary or TERT-immortalized human blood vessel endothelial cells (BECs). Thus, exposure of human blood vessel organoids to high glucose and an inflammatory milieu results in a massive thickening of the basal blood vessel membrane and altered gene expression profiles, modeling human diabetic microvasculopathy.

### Example 5

### Inhibition of γ-secretase Activity Abolishes “Diabetic” Changes in Blood Vessel Organoids

Having engineered an in vitro organoid model of human diabetic vasculopathy, we next wanted to identify a drug that could block the thickening and expansion of the basal membrane in human blood vessel organoids treated with high glucose/TNFα/IL6. To this end, we first tested multiple approved drugs that are currently used in the clinic to treat diabetes. However, none of the medicines that we tested, namely metformin, pioglitazone, glimepiride, acarbose, nateglindine, thiazolidinedione, or diphenyleneiodonium, had any effect on the high glucose/TNFα/IL6-induced thickening of the basal blood vessel membrane in blood vessel organoids (FIG. 5b,c).

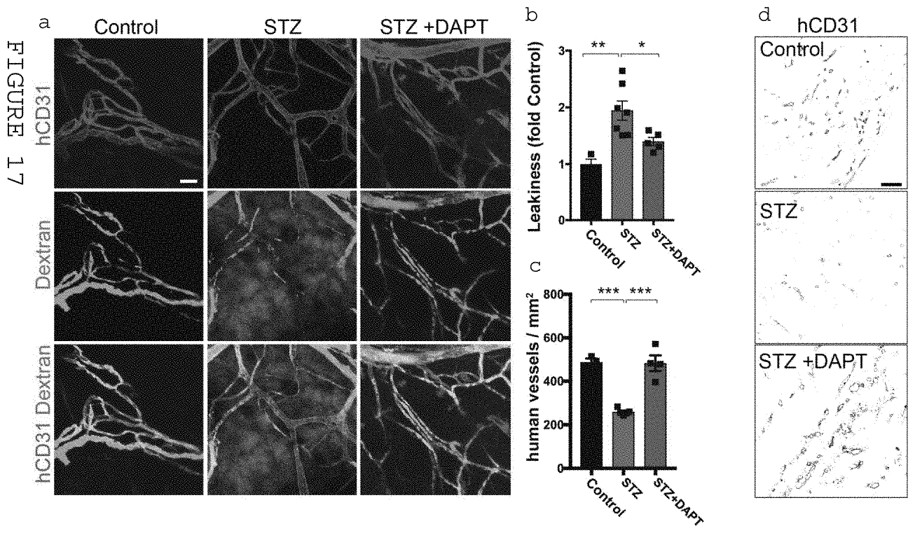

We next screened diabetic blood vessel organoids with small molecule inhibitors of various common signaling and downstream pathways, namely GSK3, PKC, AKT, NFkB, ROS, p38-MAPK, JNK, ROCK, and ERK. We found that none of these inhibitors had any significant effect on collagen IV expansion and thickening of the capillary basement membrane (FIG. 5d,e). Of note, blocking NFkB in fact markedly increased the thickening of the basal membrane. Next, we evaluated an inhibitor of γ-secretase, an enzyme that cleaves different receptors to activate distinct signaling pathways, including Notch. We found that the y-secretase inhibitor DAPT completely abrogated the expansion of collagen IV and basal membrane thickening in human blood vessel organoids exposed to the “diabetic cocktail” (FIG. 5c,d). In addition, the effects of the y-secretase inhibitor were dose-dependent (FIG. 5e), further confirming its specificity and efficacy. Importantly, we also observed that the human blood vessels become leaky in diabetic mice, providing direct evidence that the morphological changes we observe are also associated with impaired blood vessel functions; excessive blood vessel leakiness was rescued by DAPT treatment (FIG. 17a,b). Moreover, in vivo DAPT treatment rescued the loss of CD31⁺ human blood vessels in diabetic mice (FIG. 17c,d). These data show that inhibition of y-secretase activity inhibits structural as well as functional changes of diabetic blood vessels in vitro and in vivo.

### Example 6

### Identification of D114-Notch3 as Candidate Pathway for Diabetic Basal Membrane Thickening

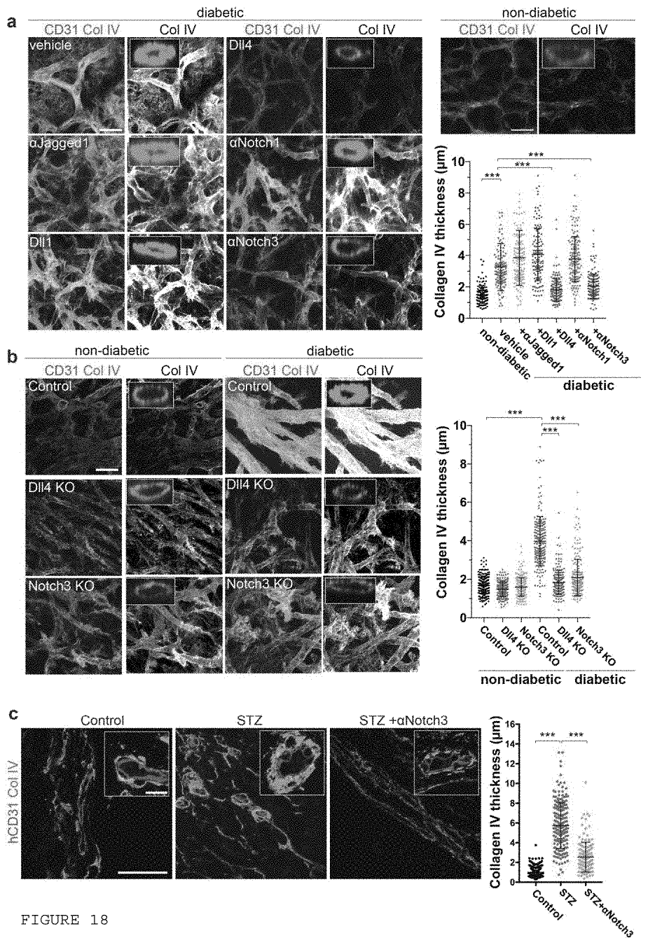

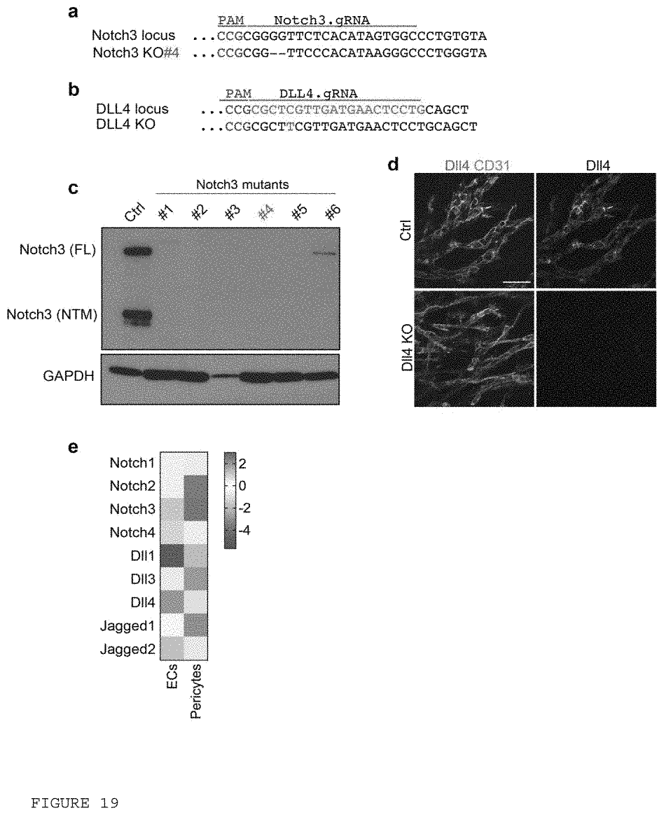

γ-secretase is an enzyme that can cleave multiple different receptors to activate distinct signaling pathways, including Notch. To identify the molecular DAPT target involved in protection from our experimental diabetic blood vessel changes, we blocked the Notch ligands Jaggedl, D111, and D114, as well as Notch1 and Notch3, all of which are prominently expressed in blood vessels. Inhibition of Jagged1, D111, as well as Notch1 had no apparent effect on the “diabetic” changes in our free-floating organoids; however, blockade of D114 as well as Notch3 significantly rescued from basal membrane thickening (FIG. 18a). To confirm these findings, we generated D114 and Notch3 mutant human iPS cells using CRISPR/Cas9 (FIG. 19a-d). From these mutant iPS cells we could readily derive vascular networks and free-floating vascular organoid (FIG. 18b). Importantly, both D114 and Notch3 mutant blood vessels exhibited a markedly reduced expansion of the basal membrane as compared to control organoids exposed to high glucose, IL6, and TNFα (FIG. 18b). Finally, in vivo treatment of STZ-treated mice carrying human blood vessel trees with an anti-Notch3 antibody showed that Notch3 blockage alleviated the basal membrane changes of human blood vessels exposed to a diabetic environment (FIG. 18c). Thus, not excluding other pathways, we have uncovered D114-Notch3 as a key ligand-receptor pair that can mediate basement membrane thickening in diabetic vasculopathy.

### CONCLUSIONS

Blood vessels contribute to the development of essentially all organ systems and have critical roles in multiple diseases ranging from strokes to heart attacks or cancer. Because of their importance, multiple cell systems have been developed to study blood vessel biology during development and disease, including the use of classic endothelial cell lines such as HUVEC cells. Moreover, endothelial cells and pericytes have each been developed from human stem cells.

We have now developed a robust and reproducible system to grow bona fide human capillaries from hESCs and iPSCs. These blood vessel organoids fulfill all previously defined criteria for human organoids. Intriguingly, blood vessel organoids transplanted into immunodeficient mice resulted in connections between the human blood vessels and the mouse circulatory system, as demonstrated by dextran perfusion, antibody injection, as well as MRI of blood flow revealing perfusion and leakage rates comparable to endogenous mouse organs. Connecting the human vasculature to the mouse circulatory system shows distinct vascular trees. Most importantly, the transplanted human organoids further develop in vivo into arterioles and venules, thus forming a true vascular tree, which has never been previously demonstrated. Therefore, these organoids could also be used to develop more complex, multi-lineage organoids, for instance form blood vessels with cardiomyocytes, or attempt to develop blood vessels in brain or liver organoids. They could also be used to study rare vascular diseases using patient-derived iPSCs.

The global prevalence of diabetes has nearly doubled in the last three decades, with a current estimate of about 420 million diabetic patients and many more with pre-diabetes, resulting in often long-term morbidities and enhanced mortality. Diabetes is a major cause of blindness, kidney failure, heart attacks, stroke and lower limb amputation, in many cases as a consequence of blood vessel pathologies such as massive thickening of the basement membrane that result in insufficient tissue oxygenation, impaired cell trafficking, or rupture of the vessels. One can model certain aspects of diabetic blood vessel changes in the retina and kidney of rodents, though until now, there was no single model that displays all the clinical features of diabetic blood vessel changes seen in human. Moreover, dermal microvascular changes do not occur in any of the diabetic rodent models we have studied, making it vital to develop novel systems to identify pathways and potential drug targets that are operational in these microvascular changes. To demonstrate the utility of our blood vessel organoids, we exposed them to a “diabetic cocktail” containing high glucose, IL6, and TNFα, that resulted in the marked expansion of the collagenous basal membrane and gene expression profiles resembling microvascular changes observed in the dermal capillaries of T2D patients.

We tested a number of current anti-diabetic medicines, as well as small molecule inhibitors of multiple common signaling pathways, but only few had any effect on the expansion of the basal membranes in diabetic organoids. Intriguingly, we found that that the γ-secretase inhibitor DAPT nearly completely prevented the thickening of the basal membrane in our organoid culture. γ-secretase inhibitors have already undergone clinical trials for Alzheimer's disease and are currently being tested as cancer treatment. These drugs can be repurposed for the treatment of diabetic vasculopathies in humans. Importantly, these data provide a proof-of-principle that the human blood vessel organoid model for diabetic microvasculopathy is a useful screening tool to discover novel drugs that alleviate microvascular changes.

γ-secretase inhibitors have already undergone clinical trials for Alzheimer's disease and y-secretase inhibitors as well as Notch2/3 blockers are currently being tested as cancer treatments. Furthermore, inhbiting the Notch pathway by y-secretase inhibitors reduced diabetes-induced glomerulosclerosis and podocyte loss by apoptosis in diabetic rats. These drugs can therefore be repurposed for the treatment of diabetic vasculopathies in humans. Importantly, these data provide a proof-of-principle that the human blood vessel organoids and our in vivo diabetic microvasculopathy model could be a useful screening tool to develop novel drugs that alleviate microvascular changes in diabetes.

1. A method of generating an artificial blood vessel organoid, comprising: providing stem cells capable of vascular differentiation; stimulating mesoderm differentiation in said stem cells; stimulating vascular differentiation in said stem cells; developing a cell aggregate from said stem cells; embedding said cell aggregate in a collagenous 3D matrix; and stimulating vascular differentiation of the aggregate in said collagenous 3D matrix.

2. The method of claim 1, wherein the cell aggregate that is embedded in a collagenous matrix comprises at least 30 cells.

3. The method of claim 1, wherein the mesoderm differentiation comprises treating the stem cells with a Wnt agonist or a GSK inhibitor, preferably CHIR99021.

4. The method of claim 1, wherein vascular differentiation in said stem cells comprises treating the stem cells with a VEGF, preferably VEGF-A, and/or a FGF, preferably FGF-2, and/or a BMP, preferably BMP4, and/or low oxygen conditions of 12% (v/v) or less atmospheric oxygen.

5. The method of claim 1, wherein vascular differentiation of the aggregate comprises treating cells of the aggregate with a VEGF, preferably VEGF-A, and/or a FGF, preferably FGF-2.

6. The method of claim 1, wherein the collagenous 3D matrix comprises at least 50 wt.-% collagen; and/or wherein the collagenous 3D matrix comprises 10%-50% laminin, 20%-70% collagen I, and/or 2%-30% collagen IV; preferably further 0.5%-10% nidogen, 0.5%-10% heparan sulfate proteoglycan, and/or 0.5%-10% entactin (all wt.-%).

7. The method of claim 1, wherein the 3D matrix is a hydrogel, preferably having a viscoelastic storage modulus G′ of 10 to 30.

8. An artificial blood vessel organoid culture comprising an interconnected network of vascular capillaries, said capillaries comprising endothelium and a basal membrane with perivascular pericytes.

9. The artificial blood vessel organoid culture of claim 8, wherein said organoid is produced by a method of generating an artificial blood vessel organoid, comprising: providing stem cells capable of vascular differentiation; stimulating mesoderm differentiation in said stem cells; stimulating vascular differentiation in said stem cells; developing a cell aggregate from said stem cells; embedding said cell aggregate in a collagenous 3D matrix; and stimulating vascular differentiation of the aggregate in said collagenous 3D matrix.

10. The artificial blood vessel organoid culture of claim 8, wherein the capillaries are embedded in an artificial 3D matrix comprising a hydrogel with collagen.

11. The artificial blood vessel organoid culture of claim 8, wherein the organoid culture comprises 40 to 1000 blood vessels as counted by counting individual vessels and vessels between capillary intersections.

12. The artificial blood vessel organoid culture of claim 8, wherein the vascular capillaries have an average diameter of from 1 μm to 30 μm; and/or wherein the ratio of endothelial cells to perivascular pericytes is between 100:1 to 1:5; and/or wherein the vascular capillaries comprise mature endothelial cells and/or mature pericytes.

13. A method of providing a non-human animal model with human vascular capillaries, wherein said human capillaries comprise endothelium and a basal membrane with perivascular pericytes, comprising the steps of: introducing a human blood vessel organoid of claim 8 into a non-human animal; and letting said organoid grow its vascular capillaries, preferably, wherein said human organoid is introduced onto or into the kidney of the non-human animal.

14. A non-human animal model comprising an inserted artificial blood vessel organoid culture according to claim 8; or a non-human animal model with human vascular capillaries, wherein said human capillaries comprise endothelium and a basal membrane with perivascular pericytes; preferably wherein the vascular capillaries of the artificial blood vessel organoid culture or the human vascular capillaries are perfused by the blood circulatory system of the non-human animal

15. The method or culture or non-human animal model of claim 1, wherein the blood vessels or capillaries are subjected to pathogenesis and said organoid or human animal model is a model of a pathology;
preferably, wherein pathogenesis comprises hyperglycemia and/or inflammation and/or wherein said pathology is diabetes, preferably wherein said inflammation comprises exposure to one or more inflammatory cytokines, preferably TNF-alpha and/or IL-6.

16. A method of screening a candidate chemical compound for influencing a pathogenesis or a pathology, comprising: administering said candidate chemical compound to a culture or non-human animal model, or during generation of said culture or non-human animal model, according to claim 1 and monitoring for physiological differences in said culture or animal model as compared to said culture or animal model without administration of the candidate chemical compound.

17. Use of an artificial blood vessel organoid according to claim 8 as an implant in a tissue replacement therapy, especially preferred a therapy comprising placing the artificial blood vessel organoid into a wound and letting said artificial blood vessel organoid culture integrate into the wound.

18. (canceled)

19. (canceled)

20. A kit suitable for the generation of an artificial blood vessel organoid according to claim 1, comprising: (i) a Wnt agonist or a GSK inhibitor; (ii) a vascular differentiation factor selected from VEGF, preferably VEGF-A, a FGF, preferably FGF-2, a BMP, preferably BMP4; (iii) a collagenous 3D matrix, preferably comprising 10%-50% laminin, 20%-70% collagen I, and/or 2%-30% collagen IV (all wt.-%).


SEQUENCE LISTING (18 sequences)

SEQ ID NO: 1 - DNA, 21 bp, Artificial Sequence
  primer
tgctgttgaaaggtgaaagag

SEQ ID NO: 2 - DNA, 18 bp, Artificial Sequence
  primer
cttggtggcgaagtctcc

SEQ ID NO: 3 - DNA, 18 bp, Artificial Sequence
  primer
acagcaaggcaacagagg

SEQ ID NO: 4 - DNA, 19 bp, Artificial Sequence
  primer
gagtaggcaggtagtccag

SEQ ID NO: 5 - DNA, 19 bp, Artificial Sequence
  primer
tcttcttttgcgtcgccag

SEQ ID NO: 6 - DNA, 17 bp, Artificial Sequence
  primer
agccccagccttctcca

SEQ ID NO: 7 - DNA, 25 bp, Artificial Sequence
  primer
caccggccactatgtgagaaccccg

SEQ ID NO: 8 - DNA, 25 bp, Artificial Sequence
  primer
aaaccggggttctcacatagtggcc

SEQ ID NO: 9 - DNA, 25 bp, Artificial Sequence
  primer
caccgcaggagttcatcaacgagcg

SEQ ID NO: 10 - DNA, 25 bp, Artificial Sequence
  primer
aaaccgctcgttgatgaactcctgc

SEQ ID NO: 11 - DNA, 21 bp, Artificial Sequence
  primer
acacaaggaaataagcaaatg

SEQ ID NO: 12 - DNA, 18 bp, Artificial Sequence
  primer
tggtcggcatcatagttc

SEQ ID NO: 13 - DNA, 20 bp, Artificial Sequence
  primer
ccagatcggtgccaagttct

SEQ ID NO: 14 - DNA, 20 bp, Artificial Sequence
  primer
gttacctgccccagactgac

SEQ ID NO: 15 - DNA, 31 bp, Artificial Sequence
  Notch 3 gRNA locus
ccgcggggttctcacatagtggccctgtgta

SEQ ID NO: 16 - DNA, 29 bp, Artificial Sequence
  Notch3 KO mutation
ccgcggttcccacataagggccctgggta

SEQ ID NO: 17 - DNA, 28 bp, Artificial Sequence
  DLL4 gRNA locus
ccgcgctcgttgatgaactcctgcagct

SEQ ID NO: 18 - DNA, 29 bp, Artificial Sequence
  DLL4 KO mutation
ccgcgcttcgttgatgaactcctgcagct